SEQUENCE LISTING (12 sequences)

SEQ ID NO: 1 - DNA, 1761 bp, Homo sapiens
ttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcattgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttccccctttttgtcccccaacttgagatgtatgaaggcttttggtctccctgggagtgggtggaggcagccagggcttacctgtacactgacttgagaccagttgaataaaagtgcacacctta

SEQ ID NO: 2 - DNA, 2210 bp, Homo sapiens
ggcacgaggcgggatttgggtcgcggttcttgtttgtggatcgctgtgatcgtcacttgacaatgcagatcttcgtgaagactctgactggtaagaccatcaccctcgaggttgagcccagtgacaccatcgagaatgtcaaggcaaagatccaagataaggaaggcatccctcctgaccagcagaggctgatctttgctggaaaacagctggaagatgggcgcaccctgtctgactacaacatccagaaagagtccaccctgcacctggtgctccgtctcagaggtgggatgcaaatcttcgtgaagacactcactggcaagaccatcacccttgaggtggagcccagtgacaccatcgagaacgtcaaagcaaagatccaggacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaagaccctgactggtaagaccatcaccctcgaggtggagcccagtgacaccatcgagaatgtcaaggcaaagatccaagataaggaaggcattcctcctgatcagcagaggttgatctttgccggaaaacagctggaagatggtcgtaccctgtctgactacaacatccagaaagagtccaccttgcacctggtactccgtctcagaggtgggatgcaaatcttcgtgaagacactcactggcaagaccatcacccttgaggtcgagcccagtgacactatcgagaacgtcaaagcaaagatccaagacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaagaccctgactggtaagaccatcaccctcgaagtggagccgagtgacaccattgagaatgtcaaggcaaagatccaagacaaggaaggcatccctcctgaccagcagaggttgatctttgccggaaaacagctggaagatggtcgtaccctgtctgactacaacatccagaaagagtccaccttgcacctggtgctccgtctcagaggtgggatgcagatcttcgtgaagaccctgactggtaagaccatcactctcgaggtggagccgagtgacaccattgagaatgtcaaggcaaagatccaagacaaggaaggcatccctcctgatcagcagaggttgatctttgctgggaaacagctggaagatggacgcaccctgtctgactacaacatccagaaagagtccaccctgcacctggtgctccgtcttagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtcttagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtctcagaggtgggatgcagatcttcgtgaagaccctgactggtaagaccatcaccctcgaggtggagcccagtgacaccatcgagaatgtcaaggcaaagatccaagataaggaaggcatccctcctgatcagcagaggttgatctttgctgggaaacagctggaagatggacgcaccctgtctgactacaacatccagaaagagtccactctgcacttggtcctgcgcttgagggggggtgtctaagtttccccttttaaggtttcaacaaatttcattgcactttcctttcaataaagttgttgcattcccaaaaaaaaaaaaaaaaaaaaaaaa

SEQ ID NO: 3 - DNA, 2201 bp, Homo sapiens
cgggatttgggtcgcggttcttgtttgtggatcgctgtgatcgtcacttgacaatgcagatcttcgtgaagactctgactggtaagaccatcaccctcgaggttgagcccagtgacaccatcgagaatgtcaaggcaaagatccaagataaggaaggcatccctcctgaccagcagaggctgatctttgctggaaaacagctggaagatgggcgcaccctgtctgactacaacatccagaaagagtccaccctgcacctggtgctccgtctcagaggtgggatgcaaatcttcgtgaagacactcactggcaagaccatcacccttgaggtggagcccagtgacaccatcgagaacgtcaaagcaaagatccaggacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaagaccctgactggtaagaccatcaccctcgaggtggagcccagtgacaccatcgagaatgtcaaggcaaagatccaagataaggaaggcattcctcctgatcagcagaggttgatctttgccggaaaacagctggaagatggtcgtaccctgtctgactacaacatccagaaagagtccaccttgcacctggtactccgtctcagaggtgggatgcaaatcttcgtgaagacactcactggcaagaccatcacccttgaggtcgagcccagtgacactatcgagaacgtcaaagcaaagatccaagacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaagaccctgactggtaagaccatcaccctcgaagtggagccgagtgacaccattgagaatgtcaaggcaaagatccaagacaaggaaggcatccctcctgaccagcagaggttgatctttgccggaaaacagctggaagatggtcgtaccctgtctgactacaacatccagaaagagtccaccttgcacctggtgctccgtctcagaggtgggatgcagatcttcgtgaagaccctgactggtaagaccatcactctcgaggtggagccgagtgacaccattgagaatgtcaaggcaaagatccaagacaaggaaggcatccctcctgatcagcagaggttgatctttgctgggaaacagctggaagatggacgcaccctgtctgactacaacatccagaaagagtccaccctgcacctggtgctccgtcttagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtcttagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtctcagaggtgggatgcagatcttcgtgaagaccctgactggtaagaccatcaccctcgaggtggagcccagtgacaccatcgagaatgtcaaggcaaagatccaagataaggaaggcatccctcctgatcagcagaggttgatctttgctgggaaacagctggaagatggacgcaccctgtctgactacaacatccagaaagagtccactctgcacttggtcctgcgcttgagggggggtgtctaagtttccccttttaaggtttcaacaaatttcattgcactttcctttcaataaagttgttgcattcccaaaaaaaaaaaaaaaaaaaaaaaa

SEQ ID NO: 4 - DNA, 25 bp, Artificial Sequence
  forward Q-PCR primer
gggtgtctaagtttccccttttaag

SEQ ID NO: 5 - DNA, 23 bp, Artificial Sequence
  reverse Q-PCR primer
ttttttgggaatgcaacaacttt

SEQ ID NO: 6 - DNA, 2310 bp, Homo sapiens
ggcacgaggggcgggactgcgcggcggcaacagcagacatgtcgggggtccggggcctgtcgcggctgctgagcgctcggcgcctggcgctggccaaggcgtggccaacagtgttgcaaacaggaacccgaggttttcacttcactgttgatgggaacaagagggcatctgctaaagtttcagattccatttctgctcagtatccagtagtggatcatgaatttgatgcagtggtggtaggcgctggaggggcaggcttgcgagctgcatttggcctttctgaggcagggtttaatacagcatgtgttaccaagctgtttcctaccaggtcacacactgttgcagcacagggaggaatcaatgctgctctggggaacatggaggaggacaactggaggtggcatttctacgacaccgtgaagggctccgactggctgggggaccaggatgccatccactacatgacggagcaggcccccgccgccgtggtcgagctagaaaattatggcatgccgtttagcagaactgaagatgggaagatttatcagcgtgcatttggtggacagagcctcaagtttggaaagggcgggcaggcccatcggtgctgctgtgtggctgatcggactggccactcgctattgcacaccttatatggaaggtctctgcgatatgataccagctattttgtggagtattttgccttggatctcctgatggagaatggggagtgccgtggtgtcatcgcactgtgcatagaggacgggtccatccatcgcataagagcaaagaacactgttgttgccacaggaggctacgggcgcacctacttcagctgcacgtctgcccacaccagcactggcgacggcacggccatgatcaccagggcaggccttccttgccaggacctagagtttgttcagttccaccccacaggcatatatggtgctggttgtctcattacggaaggatgtcgtggagagggaggcattctcattaacagtcaaggcgaaaggtttatggagcgatacgcccctgtcgcgaaggacctggcgtctagagatgtggtgtctcggtccatgactctggagatccgagaaggaagaggctgtggccctgagaaagatcacgtctacctgcagctgcaccacctacctccagagcagctggccacgcgcctgcctggcatttcagagacagccatgatcttcgctggcgtggacgtcacgaaggagccgatccctgtcctccccaccgtgcattataacatgggcggcattcccaccaactacaaggggcaggtcctgaggcacgtgaatggccaggatcagattgtgcccggcctgtacgcctgtggggaggccgcctgtgcctcggtacatggtgccaaccgcctcggggcaaactcgctcttggacctggttgtctttggtcgggcatgtgccctgagcatcgaagagtcatgcaggcctggagataaagtccctccaattaaaccaaacgctggggaagaatctgtcatgaatcttgacaaattgagatttgctgatggaagcataagaacatcggaactgcgactcagcatgcagaagtcaatgcaaaatcatgctgccgtgttccgtgtgggaagcgtgttgcaagaaggttgtgggaaaatcagcaagctctatggagacctaaagcacctgaagacgttcgaccggggaatggtctggaacacggacctggtggagaccctggagctgcagaacctgatgctgtgtgcgctgcagaccatctacggagcagaggcacggaaggagtcacggggcgcgcatgccagggaagactacaaggtgcggattgatgagtacgattactccaagcccatccaggggcaacagaagaagccctttgaggagcactggaggaagcacaccctgtcctatgtggacgttggcactgggaaggtcactctggaatatagacccgtgatcgacaaaactttgaacgaggctgactgtgccaccgtcccgccagccattcgctcctactgatgagacaagatgtggtgatgacagaatcagcttttgtaattatgtataatagctcatgcatgtgtccatgtcataactgtcttcatacgcttctgcactctggggaagaaggagtacattgaagggagattggcacctagtggctgggagcttgccaggaacccagtggccagggagcgtggcacttacctttgtcccttgcttcattcttgtgagatgataaaactgggcacagctcttaaataaaatataaatgaacaaaaaaaaaaaaaaaaaa

SEQ ID NO: 7 - DNA, 2301 bp, Homo sapiens
ggcgggactgcgcggcggcaacagcagacatgtcgggggtccggggcctgtcgcggctgctgagcgctcggcgcctggcgctggccaaggcgtggccaacagtgttgcaaacaggaacccgaggttttcacttcactgttgatgggaacaagagggcatctgctaaagtttcagattccatttctgctcagtatccagtagtggatcatgaatttgatgcagtggtggtaggcgctggaggggcaggcttgcgagctgcatttggcctttctgaggcagggtttaatacagcatgtgttaccaagctgtttcctaccaggtcacacactgttgcagcacagggaggaatcaatgctgctctggggaacatggaggaggacaactggaggtggcatttctacgacaccgtgaagggctccgactggctgggggaccaggatgccatccactacatgacggagcaggcccccgccgccgtggtcgagctagaaaattatggcatgccgtttagcagaactgaagatgggaagatttatcagcgtgcatttggtggacagagcctcaagtttggaaagggcgggcaggcccatcggtgctgctgtgtggctgatcggactggccactcgctattgcacaccttatatggaaggtctctgcgatatgataccagctattttgtggagtattttgccttggatctcctgatggagaatggggagtgccgtggtgtcatcgcactgtgcatagaggacgggtccatccatcgcataagagcaaagaacactgttgttgccacaggaggctacgggcgcacctacttcagctgcacgtctgcccacaccagcactggcgacggcacggccatgatcaccagggcaggccttccttgccaggacctagagtttgttcagttccaccccacaggcatatatggtgctggttgtctcattacggaaggatgtcgtggagagggaggcattctcattaacagtcaaggcgaaaggtttatggagcgatacgcccctgtcgcgaaggacctggcgtctagagatgtggtgtctcggtccatgactctggagatccgagaaggaagaggctgtggccctgagaaagatcacgtctacctgcagctgcaccacctacctccagagcagctggccacgcgcctgcctggcatttcagagacagccatgatcttcgctggcgtggacgtcacgaaggagccgatccctgtcctccccaccgtgcattataacatgggcggcattcccaccaactacaaggggcaggtcctgaggcacgtgaatggccaggatcagattgtgcccggcctgtacgcctgtggggaggccgcctgtgcctcggtacatggtgccaaccgcctcggggcaaactcgctcttggacctggttgtctttggtcgggcatgtgccctgagcatcgaagagtcatgcaggcctggagataaagtccctccaattaaaccaaacgctggggaagaatctgtcatgaatcttgacaaattgagatttgctgatggaagcataagaacatcggaactgcgactcagcatgcagaagtcaatgcaaaatcatgctgccgtgttccgtgtgggaagcgtgttgcaagaaggttgtgggaaaatcagcaagctctatggagacctaaagcacctgaagacgttcgaccggggaatggtctggaacacggacctggtggagaccctggagctgcagaacctgatgctgtgtgcgctgcagaccatctacggagcagaggcacggaaggagtcacggggcgcgcatgccagggaagactacaaggtgcggattgatgagtacgattactccaagcccatccaggggcaacagaagaagccctttgaggagcactggaggaagcacaccctgtcctatgtggacgttggcactgggaaggtcactctggaatatagacccgtgatcgacaaaactttgaacgaggctgactgtgccaccgtcccgccagccattcgctcctactgatgagacaagatgtggtgatgacagaatcagcttttgtaattatgtataatagctcatgcatgtgtccatgtcataactgtcttcatacgcttctgcactctggggaagaaggagtacattgaagggagattggcacctagtggctgggagcttgccaggaacccagtggccagggagcgtggcacttacctttgtcccttgcttcattcttgtgagatgataaaactgggcacagctcttaaataaaatataaatgaacaaaaaaaaaaaaaaaaaa

SEQ ID NO: 8 - DNA, 19 bp, Artificial Sequence
  forward Q-PCR primer
gggagcgtggcacttacct

SEQ ID NO: 9 - DNA, 23 bp, Artificial Sequence
  reverse Q-PCR primer
tgcccagttttatcatctcacaa

SEQ ID NO: 10 - DNA, 1142 bp, Homo sapiens
cttttccaagcggctgccgaagatggcggaggtgcaggtcctggtgcttgatggtcgaggccatctcctgggccgcctggcggccatcgtggctaaacaggtactgctgggccggaaggtggtggtcgtacgctgtgaaggcatcaacatttctggcaatttctacagaaacaagttgaagtacctggctttcctccgcaagcggatgaacaccaacccttcccgaggcccctaccacttccgggcccccagccgcatcttctggcggaccgtgcgaggtatgctgccccacaaaaccaagcgaggccaggccgctctggaccgtctcaaggtgtttgacggcatcccaccgccctacgacaagaaaaagcggatggtggttcctgctgccctcaaggtcgtgcgtctgaagcctacaagaaagtttgcctatctggggcgcctggctcacgaggttggctggaagtaccaggcagtgacagccaccctggaggagaagaggaaagagaaagccaagatccactaccggaagaagaaacagctcatgaggctacggaaacaggccgagaagaacgtggagaagaaaattgacaaatacacagaggtcctcaagacccacggactcctggtctgagcccaataaagactgttaattcctcatgcgttgcctgcccttcctccattgttgccctggaatgtacgggacccaggggcagcagcagtccaggtgccacaggcagccctgggacataggaagctgggagcaaggaaagggtcttagtcactgcctcccgaagttgcttgaaagcactcggagaattgtgcaggtgtcatttatctatgaccaataggaagagcaaccagttactatgagtgaaagggagccagaagactgattggagggccctatcttgtgagtggggcatctgttggactttccacctggtcatatactctgcagctgttagaatgtgcaagcacttggggacagcatgagcttgctgttgtacacagggtatttctagaagcagaaatagactgggaagatgcacaaccaaggggttacaggcatcgcccatgctcctcacctgtattttgtaatcagaaataaattgcttttaaagaaaaaaaaaaaaaaaaaa

SEQ ID NO: 11 - DNA, 20 bp, Artificial Sequence
  forward Q-PCR primer
gggaagatgcacaaccaagg

SEQ ID NO: 12 - DNA, 27 bp, Artificial Sequence
  reverse Q-PCR primer
tttctgattacaaaatacaggtgagga

(19) United States
(12) Patent Application Publication
(10) Pub. No.: US 2005/0014168 A1
(43) Pub. Date: Jan. 20, 2005

(54) 3' biased microarrays

(75) Inventors: Mark G. Erlander, Encinitas, CA (US); Xiao-Jun Ma, San Diego, CA (US); Thomas Baer, Mountain View, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Arcturus Bioscience, Inc., Mountain View, CA (US)

(21) Appl. No.: 10/769,476

(22) Filed: Jan. 30, 2004

Related U.S. Application Data
(60) Provisional application No. 60/475,812, filed on Jun. 3, 2003.

(51) Int. Cl. C12Q 1/68; C12M 1/34

(52) U.S. Cl. 435/6; 435/287.2

(57) ABSTRACT

The invention provides materials and methods for the detection of nucleic acid expression via the 3′ portion of expressed sequences. Embodiments of the invention include the use of microarrays comprising nucleic acid probes that are complementary to the 3′ end of expressed sequences and by the use of quantitative PCR (Q-PCR) based amplification of sequences found at or near the 3′ end of expressed sequences. The invention may be used to detect the presence of expressed nucleic acids encoding particular gene products (sequences present in a “transcriptome”).

### RELATED APPLICATIONS

This application claims benefit of priority from U.S. Provisional Patent Application Ser. No. 60/475,812, filed Jul. 3, 2003, which is hereby incorporated by reference as if fully set forth.

### TECHNICAL FIELD

The invention relates to methods and materials for the detection of nucleic acids by the use of microarrays comprising nucleic acid probes that are complementary to the 3′ end of expressed sequences and by the use of quantitative (or “real time”) PCR (“Q-PCR”) based amplification of sequences found at or near the 3′ end of expressed sequences. The probes of the microarrays are short oligonucleotides that may be used to detect the presence of expressed nucleic acids encoding particular gene products (sequences present in a “transcriptome”). The primers and optional probes for Q-PCR are also short oligonucleotides that may be used to detect the presence of expressed nucleic acid sequences present in a transcriptome. The probes and primers are also particularly useful for distinguishing the expressed forms of different members of a gene family as well as for the detection of the expression levels of reference gene sequences. Methods for the design and use of the microarrays of the invention, along with the design and use of the primers for Q-PCR, are also provided.

### BACKGROUND ART

The ability to use microarrays in gene expression analysis is affected by sequence selection, probe selection, and array design, which all relate to the physical microarray which will be used to generate data for analysis by an algorithm of choice. With the availability of the genomes of various organisms, the ability to conduct gene expression analysis on those organisms is even more affected by sequence selection and probe selection, especially the latter where the expression of all sequences of a genome is to be analyzed.

Probe selection provides a particularly unique set of challenges. Aside from the overarching need to select probe sequences with similar hybridization characteristics, there is the need to select probe sequences that are unique to particular gene sequences (or the consensus sequences thereof) to maximize accuracy by having each positive hybridization event being definitive for the expression of one gene sequence. This is particularly evident in the case of members of a gene family, where there are significant similarities in the gene sequences encoding different members of the family. There is also the need to provide redundancy by selecting more than one probe sequence that is unique to each gene sequence (or the consensus sequence thereof) so that each positive hybridization event may be corroborated by another to definitively identify the expression of a gene sequence. The use of consensus sequences is necessary in part to reduce the effect of ambiguous and polymorphic bases to permit the selection of probe sequences that are capable of hybridizing to the same expressed gene from different individual organisms.

Therefore, probe sequences have been selected from the entirety length of a gene sequence (or the consensus sequence thereof) to provide increased ability to select probe sequences with similar hybridization characteristics, probe sequences that are unique to particular gene sequences, multiple probe sequences for each gene sequence, and probes that will detect gene expression from multiple individuals. The use of the entire length of a gene sequence (or the consensus sequence thereof) also provides for the possibility of selecting probe sequences that would be able to distinguish between alternate splice forms that occur with the expression of a particular genomic sequence.

The above advantages of using the entire length of gene sequences would be reduced or lost if probe selection were limited to particular regions of gene sequences.

PCR is a laboratory method for the exponential amplification of nucleic acid molecule. Reverse transcription PCR is a related method for the amplification of single stranded RNA. Either form of PCR may be used with nucleic acids such as that found in a biological sample or with nucleic acids that have been derived or amplified from a biological sample. PCR may also be conducted quantitatively (or in “real time”) by the use of a set of primers and a fluorogenic probe. Quantitative PCR (Q-PCR) refers to the ability to monitor the progress of the PCR reaction, usually by fluorometric means as the reaction progresses. Q-PCR allows quantitative measurements of RNA (or DNA) to be made with much more precision and reproducibility because it relies on threshold cycle (CT) values determined during the exponential phase of PCR rather than endpoint measurements.

One type of Q-PCR uses a primer pair with a fluorogenic (dark-hole-quencher) probe and is based on the hydrolysis of the fluorogenic probe. The probe, containing a 5′-fluorophore and a 3′-quencher, anneals to a specific target sequence between the upstream and the downstream primers of a PCR reaction. To prevent its use as a primer, the 3′-terminus of the probe may be optionally blocked with PO₄, NH₂ or other blocked base. Under appropriate cycling conditions, the PCR reaction proceeds as the 5′ to 3′-endonuclease activity of the thermal stable polymerase enzyme cleaves and releases the fluorophore from the probe. After release, the fluorophore is no longer in close proximity to the quencher, and thus the fluorescence becomes detectable. As the concentration of released fluorophore in solution increases, the resultant fluorescent signal is monitored by real-time fluorometric analysis.

Fluorescence values may be recorded during every PCR cycle. The values represent the amount of product amplified to that point in the amplification reaction. Increased numbers of templates present at the beginning of the reaction permits fewer PCR cycles to reach a point in which the fluorescence signal is first detectable as statistically significant above background, which defines the Ct value for each cycle.

### DISCLOSURE OF THE INVENTION

The present invention is based in part on the observation that gene expression analysis is improved by detection of nucleic acid sequences present at the 3′ end of expressed genes. Therefore, the invention provides for the use of microarrays comprising probe sequences from the 3′ end of gene sequences. The invention also provides for the use of quantitative PCR (Q-PCR) for the detection of expressed sequences present at the 3′ end of expressed gene transcripts.

The invention is also based in part on the discovery that the 3′ region of gene sequences from an organism contains unique sequences sufficient to permit expression analysis of different members of a gene family. Therefore, the invention provides for probes which are capable of hybridizing to one or more of those unique sequences as well as Q-PCR primers and optional probes for detecting the presence of such unique sequences.

Therefore in a first aspect, the invention thus provides for microarrays containing oligonucleotide probes that contain sequences that are found less than 360 nucleotides from the polyadenylation site of polyadenylated mRNA transcripts (or their cDNA counterparts). The probes are selected to be capable of hybridizing to the mRNA transcripts (or their cDNA or amplified RNA counterparts) to serve as a means to detect the presence of the transcripts. The microarrays of the invention may contain as many probes as are desired as long as it also contains probes from the region within 360 nucleotides of the polyadenylation site of the mRNA transcripts (or their cDNA or amplified RNA counterparts) to be detected.

In this aspect of the invention, a microarray comprising at least 5 probes is provided. Each probe is about 150 nucleotides or less in length, and each probe is complementary to at least 10 consecutive nucleotides of an mRNA molecule (or its cDNA counterpart) wherein said at least 10 consecutive nucleotides is, in its entirety, less than 360 nucleotides from the site of poly(A) addition of said mRNA molecule. Stated differently, a microarray of the invention comprises 10 or more oligonucleotide probes such that at least 90% of said probes are as described above.

In some embodiments of the invention, the microarrays of the invention comprise at least 10, 20, 30, 40, 50, 60, 80, or 100 probes as described above.

In other embodiments of the invention, the at least 10 consecutive nucleotides of the probes is, in its entirety, less than about 340, less than about 320, less than about 300, less than about 280, less than about 260, less than about 240, less than about 220, less than about 200, less than about 180, less than about 160, less than about 140, less than about 120, less than about 100, less than about 80, less than about 60, or less than about 50, nucleotides from the polyadenylation site of mRNA transcripts (or their cDNA or amplified RNA counterparts) to be detected. The term “about” as used in this paragraph encompasses the presence or absence of approximately 10 or less nucleotides.

In a second aspect, the invention provides compositions and methods for Q-PCR based detection of sequences present less than 360 nucleotides from the polyadenylation site of polyadenylated mRNA transcripts (or their cDNA counterparts). The compositions and methods may be used to quickly detect the presence of expressed transcripts in a biological sample, either directly or after the amplification of the transcripts. Using primers and optional probes specific to the 3′ region, the methods include amplifying and monitoring the development of specific amplification products using Q-PCR. Preferably, the primers amplify a sequence comprising at least 10 consecutive nucleotides of an mRNA molecule (or its cDNA counterpart) wherein said at least 10 consecutive nucleotides is, in its entirety, less than 360 nucleotides from the site of poly(A) addition of said mRNA molecule. In other embodiments, the at least 10 consecutive nucleotides of the probes is, in its entirety, less than 340, 320, 300, 280, 260, 240, 220, 200, 180, 160, 140, 120, 100, 80, 75, 70, 65, 60, 55, 50, 40, 30, 20, or 10 nucleotides from the polyadenylation site of mRNA transcripts (or their cDNA or amplified RNA counterparts) to be detected. The optional probe hybridizes to (targets) an amplified sequence, which is within 360 nucleotides of the polyadenylation site. One or both of the primers may be more than 360 nucleotides from the polyadenylation site.

In this aspect of the invention, an assay method for detecting the presence or absence of an expressed sequence in a biological sample from an individual includes performing at least one cycling step, which includes a nucleic acid amplification step and a hybridization step. The amplifying step includes contacting a sample with at least a pair of Q-PCR primers to produce an amplification product if the sequence to be amplified is present in the sample, and the hybridizing step includes contacting the sample with at least one Q-PCR probe which hybridizes to a sequence in the amplified product. Preferably, the expressed sequence to be analyzed is one correlated with disease or an unwanted condition by virtue of increased or decreased expression.

Alternatively, the expressed sequence to be analyzed may be one used as a “reference” expressed sequence for determination of relative expression levels of another expressed sequence, such as one associated with a disease or unwanted condition. Preferred reference sequences of the invention are those that have the same or similar levels of expression in both normal and abnormal (or non-normal cells), including, but not limited to non-cancer (or non-tumor) and cancer (or tumor) cells. The expression level of one or more reference sequence may be used in comparison to the expression level of an expressed sequence correlated with disease or an unwanted condition by virtue of increased or decreased expression. In preferred embodiments, the expression levels of both the reference sequence and the sequence correlated with disease or unwanted condition are determined using the same cell. Non-limiting examples of such cells include those from a cell containing sample from a subject afflicted with, or suspected of being afflicted with, the disease or unwanted condition or otherwise as described herein.

With probe hydrolysis based Q-PCR as disclosed herein, the at least one Q-PCR probe preferably hybridizes to a sequence within the region amplified by a pair of Q-PCR primers. This may be the case even where the probe is complementary to a portion of one of the two primers (e.g. where the 3′ portion of a probe is complementary to the 3′ portion of a primer). A Q-PCR probe is typically labeled with a donor fluorescent moiety and a second quencher or acceptor fluorescent moiety. The detection methods of the invention further include detecting the presence or generation of detectable fluorescence, and thus the absence or decrease in fluorescence resonance energy transfer (FRET) between the donor fluorescent moiety and the quencher or acceptor fluorescent moiety in the Q-PCR probe. The presence or generation of detectable fluorescence is indicative of the presence of an expressed sequence in the biological sample, and the absence of detectable fluorescence is indicative of the absence of an expressed sequence in the biological sample.

Fluorescence is preferably detected by using a (thermostable) polymerase enzyme having 5′ to 3′ exonuclease activity which cleaves the donor fluorescence moiety from the probe to result in a detectable signal during amplification. The donor and quencher or acceptor moieties on the probe are preferably located such that FRET may occur between the two moieties. In some embodiments, the location of the donor moiety at or near the 5′ end of the probe and the quencher or acceptor moiety at or near the 3′ end of the probe with a separation of from about 14 to about 22 basepairs between the moieties, although other distances, such as from about 6, about 8, about 10, or about 12 basepairs may be used. Preferred distances are about 14, about 16, about 18, about 20, or about 22 basepairs. In another form of such a method, the Q-PCR probe can include a nucleic acid sequence that permits secondary structure formation (such as a hairpin) that results in spatial proximity between the donor and the quencher or acceptor fluorescent moiety. Such a method does not require hydrolysis of the probe and has been referred to as the “molecular beacon” approach (see for example, Tyagi S et al. (1996) Molecular beacons: probes that fluoresce upon hybridization. Nat Biotechnol 14, 303-308).

In yet another alternative form of the invention, a method is provided for detecting the presence or absence of an expressed sequence in a biological sample from an individual as described above except for the use of a pair of probes where one probe contains the donor moiety and the other probe contains the acceptor moiety. Such a method still includes performing at least one cycling step, wherein a cycling step comprises amplification and hybridization. The amplifying step still includes contacting the sample with a pair of Q-PCR primers to produce an amplification product if the expressed sequence to be amplified is present in the sample. The hybridizing step includes contacting the sample with a pair of probes as described above. The method further includes detecting the presence or absence of fluorescence resonance energy transfer (FRET) between the donor fluorescent moiety and the acceptor fluorescent moiety of the two probes. The presence or absence of FRET is indicative of the presence or absence of the expressed sequence in the sample. Such a method can optionally further include determining the melting temperature between the amplification product and one or both of the probes. The melting temperature can confirm the presence or absence of the expressed sequence.

In a further alternative form of the invention, a method is provided for detecting the presence or absence of an expressed sequence in a biological sample from an individual as described above except for the use of a nucleic acid binding dye in place of any nucleic acid probe. Such a method still includes performing at least one cycling step, wherein a cycling step comprises amplification and a dye-binding step. The amplifying step includes contacting the sample with a pair of Q-PCR primers to produce an amplification product if the expressed sequence to be amplified is present in the sample. The dye-binding step comprises contacting the amplification product with a nucleic acid binding dye. The method further includes detecting the presence or absence of binding of the nucleic acid binding dye to the amplification product. The presence of binding is usually indicative of the presence of the expressed sequence in the sample, and the absence of binding is usually indicative of the absence of the expressed sequence in the sample. Non-limiting examples of nucleic acid binding dyes include SybrGreen I®, SybrGold®, and ethidium bromide. Such a method can further include determining the melting temperature between the amplification product and the nucleic acid binding dye. The melting temperature can confirm the presence or absence of an expressed sequence.

Representative donor fluorescent moieties for use in the present invention include, but are not limited to, FAM or 6-FAM, fluorescein, HEX, TET, TAM, ROX, Cy3, Alexa, and Texas Red while non-limiting examples of a quencher or acceptor fluorescent moiety include MGB, TAMRA, BHQ (black hole quencher), LC™-RED 640 (LightCycler™-Red 640-N-hydroxysuccinimide ester), LC™-RED 705 (LightCycler™-Red 705-Phosphoramidite), and cyanine dyes such as CY5 and CY5.5. As will be appreciated by a person skilled in the art, any pair of donor and quencher/acceptor moieties may be used as long as they are compatible such that transmission may occur from the donor to the quencher/acceptor. Moreover, pairs of suitable donors and quenchers/acceptors are known in the art and are provided herein. The selection of a pair may be made by any means known in the art and may be confirmed by routine and repetitive testing for energy transfer or quenching of fluorescence.

A pair of Q-PCR primers generally includes a first primer and a second primer. The first and second primers can contain sequences as described herein or sequences capable of serving as primers for amplification of sequences from within the 3′ end of expressed sequences. Preferably, and in the practice of probe hydrolysis based embodiments of the invention, the primers are no more than about 150 basepairs from the probe for improved sensitivity in detecting Q-PCR amplified sequences.

In some practices of the invention, the detecting step includes exciting the combination of nucleic acid material (such as transcripts, or amplified versions thereof, from a biological sample), primer, and probe with a wavelength absorbed by the donor fluorescent moiety and detecting, visualizing and/or measuring fluorescence released from the donor moiety. The amount of detectable fluorescence will depend upon the proximity of the donor moiety to the quencher or acceptor fluorescent moiety. In another aspect, the detecting step is performed after each cycling step, and further, can be performed in real-time. In an alternative aspect, the detecting may comprise quantitating the FRET to the quencher or acceptor fluorescent moiety. The assay methods of the invention are platform independent and work well on at least instrument that support fluorogenic probe hydrolysis assays, including the ABI 7700, the Cepheid Smart Cycler and the Roche Light Cycler.

Generally, the presence of fluorescence in less than about 50 cycles, in less than about 45 cycles, in less than about 40 cycles, in less than about 35 cycles, in less than about 30 cycles, in less than about 25 cycles, or in less than about 20 cycles, indicates the presence of an expressed sequence that has been amplified by the Q-PCR reaction in the individual from which the sample was obtained.

The methods of the invention can further include amplification of a control nucleic acid. The cycling step can be performed on a control sample. A control sample can include a control nucleic acid molecule. Alternatively, such a control sample can be amplified using a pair of control primers and hybridized to a control probe. The control primers and the control probe are usually other than the primers and the probe(s) used to amplified a sequence to be detected. A control amplification product is produced if control template is present in the sample, and the control probes hybridize to the control amplification product.

In other embodiments, the invention may be practiced in a manner to prevent or decrease amplification of contaminating nucleic acids in a sample. Non-limiting examples of such means include the use of uracil-DNA glycosylase as described in U.S. Pat. Nos. 5,035,996, 5,683,896 and 5,945,313 to reduce or eliminate contamination between one thermocycler run and the next.

In general, the use of a probe sequence, or Q-PCR primers, complementary to a sequence less than 360 nucleotides upstream (i.e. in the 5′ direction) from the polyadenylation site of an mRNA transcript (or its cDNA or amplified RNA counterparts) would be expected to result in disadvantages. One disadvantage is that the ability to differentiate splice variants (mRNA transcripts that result from alternative splicing events) is lost for variants where the difference in sequence is not within the region complementary to the probe sequence.

However, splice variants with differences in sequence within the region complementary to the probes or Q-PCR primers of the invention, or splice variants that result in different polyadenylation sites, may still be differentiated by detection of hybridization to probes of the invention.

The microarrays and Q-PCR based reactions of the invention may be used in methods to conduct quantitative and qualitative analysis of gene expression. Stated differently, the microarrays and Q-PCR methods may be used to detect expression of sequences found in the transcriptome of a particular cell, tissue, organ, or subject. Preferably, the expressed gene sequences are those encoded by the human genome and/or human mitochondrial genome. Thus the invention provides for methods of identifying or detecting or quantifying the expression of various gene sequences by use of the microarrays or Q-PCR methods described herein. The invention may be used upon the induction of gene expression in a cell, tissue, organ, or subject. Alternatively, the invention may be used to study gene expression as the result of a disease state in a cell, tissue, organ, or subject. Particularly, the expression of genes in cells that are not normal, pre-cancerous, cancerous, or invasive (such as, but not limited to, breast cancer) may be identified, detected or quantified. Similarly, the methods may be used to identify, detect, or quantify gene expression during differentiation at the cellular, tissue, or organ level.

The microarrays and Q-PCR based methods may also be used in the study of functional gene networks. The invention thus provides for methods of identifying or detecting the expression of various gene sequences to define or identify gene networks by use of the microarrays and Q-PCR methods described herein. These methods may also be used to identify networks that are involved in cancer or tumorigenesis or during differentiation.

In another aspect of the invention, there are provided articles of manufacture beyond microarrays, comprising pairs of Q-PCR primers and optional Q-PCR probes with a donor fluorescent moiety and a corresponding quencher or acceptor moiety. The probes in such articles of manufacture or kits can be labeled with a donor fluorescent moiety and with a corresponding quencher or acceptor fluorescent moiety. The articles of manufacture or kits may also optionally include a package label or package insert having instructions thereon for use in a Q-PCR method of the invention.

The details of one or more embodiments of the invention are set forth in the description below.

### MODES OF CARRYING OUT THE INVENTION

Definitions

An “oligonucleotide” is a type of “polynucleotide,” which is a polymeric form of nucleotides of any length, either ribonucleotides or deoxyribonucleotides. This term refers only to the primary structure of the molecule. Thus, this term includes double- and single-stranded DNA and RNA, although single stranded probes are preferred for the microarrays, and Q-PCR primers and probes, of the invention. “Oligonucleotide” refers to polynucleotides of a relatively shorter length. An oligonucleotide of the invention may comprise modifications, including labels, known in the art. Non-limiting examples include methylation, substitution of one or more of the naturally occurring nucleotides with an analog, internucleotide modifications such as uncharged linkages (e.g., phosphorothioates, phosphorodithioates, etc.), and modified linkages (e.g., alpha anomeric nucleic acids, etc.), as well as unmodified forms. The scope of oligonucleotide as used in the context of the invention may be functionally defined by its ability to hybridize to an mRNA transcript (or its cDNA or amplified RNA counterparts).

The term “amplify” as in “amplified RNA” is used in the broad sense to mean creating an amplification product which may contain all or part, or be complementary to all or part, of a nucleic acid molecule. An amplification product can be made enzymatically with DNA or RNA polymerases, such as PCR based and in vitro transcription (IVT) based amplification, respectively. “Amplification,” as used herein, generally refers to the process of producing multiple copies of a desired sequence. “Multiple copies” mean at least 2 copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

A “microarray” is a linear or two-dimensional array of preferably discrete regions, each having a defined area, formed on the surface of a solid support. The density of the discrete regions on a microarray is determined by the total numbers of target polynucleotides to be detected on the surface of a single solid phase support, preferably at least about 50/cm², more preferably at least about 100/cm², even more preferably at least about 500/cm², and still more preferably at least about 1,000/cm². As used herein, a DNA microarray is an array of oligonucleotide probes placed on a chip or other surfaces used to hybridize to target polynucleotides of interest, such as mRNA transcripts (or their cDNA or amplified RNA counterparts). Since the position of each particular probe in the array is known, the identities and amount of the target polynucleotides can be determined based on their binding to a particular position in the microarray.

The term “label” refers to a composition capable of producing a detectable signal indicative of the presence of the target polynucleotide in an assay sample. Suitable labels include radioisotopes, nucleotide chromophores, enzymes, substrates, fluorescent molecules, chemiluminescent moieties, magnetic particles, bioluminescent moieties, and the like. As such, a label may be considered as any composition detectable by spectroscopic, photochemical, biochemical, immunochemical, electrical, optical or chemical means.

Polynucleotides for hybridization to the microarrays of the invention, or subjected to Q-PCR as described herein, may be obtained from a biological sample or by amplification from such a sample. As used herein, a “biological sample” refers to a sample of tissue or fluid isolated from an individual, including but not limited to, for example, blood, plasma, serum, spinal fluid, lymph fluid, fine needle aspirates (FNA), collections from ductal lavage, the external sections of the skin, respiratory, intestinal, and genitourinary tracts, tears, saliva, milk, cells (including but not limited to blood cells), tumors, organs, and also samples of in vitro cell culture constituents.

A “portion” or “region,” used interchangeably herein, of a polynucleotide or oligonucleotide is a contiguous sequence of 2 or more bases. It may also be considered a region or portion is at least about any of 3, 5, 10, 15, 20, 25 contiguous nucleotides.

“Expression” includes transcription and/or translation, although the microarrays and Q-PCR based methods of the invention are designed to detect nucleic acid transcripts as opposed to translation products.

“Transcriptome” refers to the transcribed fraction and/or the transcribed form(s) of the genes in the genome of a cell, tissue, organ, or organism.

As used herein, the term “comprising” and its cognates are used in their inclusive sense; that is, equivalent to the term “including” and its corresponding cognates.

Conditions that “allow” an event to occur or conditions that are “suitable” for an event to occur, such as hybridization, strand extension, and the like, or “suitable” conditions are conditions that do not prevent such events from occurring. Thus, these conditions permit, enhance, facilitate, and/or are conducive to the event. Such conditions, known in the art and described herein, depend upon, for example, the nature of the nucleotide sequence, temperature, and buffer conditions. These conditions also depend on what event is desired, such as hybridization, cleavage, strand extension or transcription.

The term “3′” (three prime) generally refers to a region or position in a polynucleotide or oligonucleotide 3′ (downstream) from another region or position in the same polynucleotide or oligonucleotide.

The term “5′” (five prime) generally refers to a region or position in a polynucleotide or oligonucleotide 5′ (upstream) from another region or position in the same polynucleotide or oligonucleotide.

The term “3′-DNA portion,” “3′-DNA region,” “3′-RNA portion,” and “3′-RNA region,” refer to the portion or region of a polynucleotide or oligonucleotide located towards the 3′ end of the polynucleotide or oligonucleotide, and may or may not include the 3′ most nucleotide(s) or moieties attached to the 3′ most nucleotide of the same polynucleotide or oligonucleotide. The 3′ most nucleotide(s) can be preferably from about 1 to about 20, more preferably from about 3 to about 18, even more preferably from about 5 to about 15 nucleotides.

The term “5′-DNA portion,” “5′-DNA region,” “5′-RNA portion,” and “5′-RNA region,” refer to the portion or region of a polynucleotide or oligonucleotide located towards the 5′ end of the polynucleotide or oligonucleotide, and may or may not include the 5′ most nucleotide(s) or moieties attached to the 5′ most nucleotide of the same polynucleotide or oligonucleotide. The 5′ most nucleotide(s) can be preferably from about 1 to about 20, more preferably from about 3 to about 18, even more preferably from about 5 to about 15 nucleotides.

“Detection” includes any means of detecting, including direct and indirect detection. For example, “detectably fewer” products may be observed directly or indirectly, and the term indicates any reduction (including no products). Similarly, “detectably more”-product means any increase, whether observed directly or indirectly.

Polyadenylation site refers to the nucleotide to which a polyadenylate tail is attached. The site may be readily identified empirically, such as by examination of a sequence to determine where a poly A tract (or a complementary poly T tract) begins. The amount of interruption within a tract maybe used by a skilled person to determine whether a poly A tail is present. The polyadenylation site location can also be supported by examination of the sequence 5′ from the site to identify a polyadenylation signal, such as the AAUAA sequence found from 11 to 30 nucleotides upstream of poly(a) addition in polyadenylated mRNA of higher eukaryotes, consistent with the site's location. Alternatively, the polyadenylation site may be defined as a nucleotide position within a particular distance from a polyadenylation signal, such as from 11 to 30 nucleotides downstream from an AAUAA sequence of an mRNA (or its cDNA or amplified RNA counterparts). This can be supported by the polyadenylation signal (e.g. AAUAA) being downstream (3′ of) the coding region of the mRNA (or its cDNA or amplified RNA counterparts) and/or the absence of any 3′ untranslated sequence of the mRNA in the region of 11 to 39 nucleotides downstream of the signal.

For sequences lacking a poly A (or complementary poly T) tract, the last 3′ nucleotide position may be treated as the polyadenylation site until the actual polyadenylation site for the sequence is identified. Where alternate polyadenylation sites are identified for the same sequence, such as in the case of splice variants with different polyadenylation sites, either or both may be used as the polyadenylation site for the determination of the region to which probes of the invention are complementary.

As used in this specification and the appended claims, the singular forms “a”, “an” and “the” include corresponding plural references unless the context clearly dictates otherwise.

Unless defined otherwise all technical and scientific terms used herein have the same meaning as commonly understood to one of ordinary skill in the art to which this invention belongs.

General Methods

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., eds., 1994).

Probes, oligonucleotides and polynucleotides employed in the present invention can be generated using standard techniques known in the art.

Microarray Related Embodiments of the Invention

In a first aspect, the present invention is directed to microarrays containing probe sequences with a bias toward hybridization to the 3′ end (or region) of expressed gene sequences of a cell. The probes of the microarrays are preferably single stranded oligonucleotides in nature, and may be at least about 20, about 25, about 30, about 40, about 50, about 60, about 70, about 80, about 90, about 100, about 110, about 120, about 130, about 140, or about 150 nucleotides in length. Preferred lengths are 30, 60, 90, 100, 120, and 150 nucleotides, although lengths of 20 or 25 may also be used. The microarrays of the invention contain at least 5 probes, preferably, at least 10, 20, 30, 40, 50, 60, 80, 100, 150, 200, 250, 300, 350, 400, 450, 500, 600, 700, 800, 900, 1000, 1500, 2000, 2500, 3000, 4000, or 5000 probes. In some embodiments of the invention, the arrays contain less than 5000, 4000, 3000, 2000, or 1000 probes. They range from at least 10, 20, 30, 40, 50, 60, 80, 100, 150, 200, 250, 300, 350, 400, 450, or 500 probes to 1000, 2000, 3000, 4000 or 5000 probes.

An oligonucleotide probe of the invention contains at least 10 consecutive nucleotides which are, in their entirety, less than 360 nucleotides from the polyadenylation site of an mRNA molecule (or its cDNA or amplified RNA counterparts). The sequence that is less than 360 nucleotides from the polyadenylation site may be wholly or partly the 3′ untranslated region of the mRNA (or its cDNA or amplified RNA counterparts) or alternatively be wholly or partly within the 3′ coding region of the mRNA (or its cDNA or amplified RNA counterparts). Preferably, at least 15, 20, 25, 30, 35, 40, 45, 50, 55, 60, 65, 70, 75, 80, 85, 90, 95, or 100 consecutive nucleotides of a probe of the invention are complementary to a sequence less than 360 nucleotides from the polyadenylation site of an mRNA molecule (or its cDNA or amplified RNA counterparts). Of course a probe that is complementary, in its entire length, to a sequence less than 360 nucleotides from the polyadenylation site of an mRNA molecule (or its cDNA or amplified RNA counterparts) is within the scope of the invention.

The at least 10 consecutive nucleotides of the probes may, in its entirety, be complementary to a sequence less than 340, 320, 300, 280, 260, 240, 220, 200, 180, 160, 140, 120, 100, 80, 60, 50, 40, 30, 20, or 10 nucleotides upstream from (or 5′ of) the polyadenylation site of mRNA transcripts (or their cDNA or amplified RNA counterparts) to be detected.

The invention thus provides a microarray comprising at least 5 probes, each probe being about 150 nucleotides or less in length, and each probe being complementary to at least 10 consecutive nucleotides of an mRNA molecule wherein said at least 10 consecutive nucleotides is, in its entirety, less than 360 nucleotides from the site of poly(A) addition of said mRNA molecule (or its cDNA or amplified RNA counterparts).

The microarrays of the invention may also be defined in terms of their percent composition of oligonucleotide probes as described above. Preferably, a microarray of the invention comprises 10 or more oligonucleotide probes wherein at least 80, 85 or 90% of said probes are as described above. In some embodiments of the invention at least 80, 85 or 90% of said probes of the microarray are as described above.

The microarrays of the invention may also comprise probes that hybridize to normalization control gene sequences. These probes need not be defined as provided above, but rather need only be selected to hybridize to gene sequences that are expressed with relatively low signal variation over different samples. For example, gene sequences that are expressed at relatively constant levels in breast cells or tissue under a variety of conditions may be used for the selection of probes that hybridize to mRNA transcripts of such sequences. The expression levels of these transcripts may be used to scale data concerning the expression of other gene sequences to reduce or eliminate data skewing.

Preparation of the Microarrays

The microarrays of the invention maybe prepared by standard methods known in the art for microarrays containing oligonucleotide probes. Several techniques are well-known in the art for attaching nucleic acids to a solid substrate such as a glass slide. One method is to incorporate modified bases or analogs that contain a moiety that is capable of attachment to a solid substrate, such as an amine group, a derivative of an amine group or another group with a positive charge, into the amplified nucleic acids. The oligonucleotide probe is then contacted with a solid substrate, such as a glass slide, which is coated with an aldehyde or another reactive group which will form a covalent link with the reactive group that is on the amplified product and become covalently attached to the glass slide.

Non-limiting examples include the preparation of arrays using polynucleotides that have been amino-modified at a 5′-terminus by using a 5′-amino-modified primer, such as via PCR amplification. A 5′-amino-modified PCR product can be attached to a microscope slide or other solid surface which has been derivatised with an aldehyde group. Formation of a covalent bond between the amino group on the polynucleotide and the aldehyde group provides a permanent attachment to the slide or other solid surface.

Similarly, and to produce oligonucleotide arrays, many oligonucleotides are synthesized using standard DNA solid phase synthesizers with 5′-amino- or thio-modifications of the oligonucleotides during synthesis. The 5′ modification may be added directly to the oligonucleotide during synthesis or indirectly by incorporating a long linker between the amino or thio group and the 5′-end of the oligonucleotide sequence itself. The linker may be part of the phosphoramidite used in the synthesis of the oligonucleotide or a separate linker phosphoramidite that is inserted between the last base of the sequence and the amino or thiol reactive group. A long linker, such as but not limited to a C12 or longer linker may be added to connect the reactive group to the oligonucleotide. The use of a linker or other means to distance the oligonucleotide from the surface of the microarray permits maximization of hybridization between the probe and its target polynucleotide by distancing the oligonucleotide from the microarray surface.

Other methods for in situ oligonucleotide synthesis on microarrays. One method is the photolithography method, which uses phosphoramidite chemistry to link free hydroxy groups on a glass slide or other solid surface with a linker containing a photo-labile blocking group (e.g. MeNPOC or [R,S]-1-[3,4-[methylene-dioxy]-6-nitrophenyl]ethyl chloroformate). The photo-labile blocking group is then selectively removed from defined locations on the microarray surface by shining light through a mask onto the locations on the microarray surface. The first base of the oligonucleotide sequence is introduced by reacting the 3′ hydroxyl group of the incoming 5′-photo-labile-blocked nucleoside phosphoramidite with the available de-blocked positions on the microarray slide. Applications of other masks to remove the photo-labile group from other selected locations using light, each of the other three 5′-photo-labile blocked nucleoside phosphoramidites may be introduced at defined locations to complete attachment of the first nucleotide of all oligonucleotides on the microarray. The addition of additional nucleotides can be achieved by use of other masks and 5′-photo-labile blocked nucleoside phosphoramidites as needed to produce oligonucleotides in a 3′ to 5′ direction. While this approach permits a very high density of oligonucleotides on a microarray, it has a disadvantage in that the overall efficiency in each cycle is low. A variation of the above removes the need for masks by using computer-controlled micromirror arrays to direct the light to desired locations on a microarray.

Another in situ synthesis method for oligonucleotide microarrays uses ink-jet style synthesis with standard dimethoxytrityl blocked phosphoramidites. The step wise coupling efficiency is higher than seen with the photolithography method above. The quality of longer oligonucleotides produced on the microarrays is thus better. This approach may also utilize reverse amidites (3′-dimethoxytrityl-blocked 5′-phosphoramidites rather than 5′-dimethoxytrityl-blocked 3′-phosphoramidites) to make oligonucleotides in the 5′ to 3′ direction to result in free 3′-OH groups.

Other methods are known, such as those using amino propyl silicon surface chemistry and those attaching PCR amplified polynucleotides onto surfaces pre-coated with poly-L-lysine. Attachment of groups to the probes, as arrayed above, which could be later converted to reactive groups is also possible using methods known in the art.

The probe sequences used on the microarrays of the invention may be selected based upon sequences from publicly available sources, such as GenBank, dbEST, RefSeq, Washington University EST trace repository, and University of Santa Cruz golden-path human genome database. The sequence from these sources may also be supplemented by any other sequence information as desired by a skilled person in the field. The use of EST sequences may be preceded by analyzing them for untrimmed, low-quality sequence information, correct orientation, false priming, false clustering, and alternative splicing followed by correction or removal of sequences from consideration as known in the art. EST sequences may also be analyzed for alternative polyadenylation to confirm the existence of, and identify the location of, more than one polyadenylation site.

The probe sequences may also be selected after analysis of sequence clusters, such as those of UniGene, and/or with genome based subclustering. The use of genome based subclustering is particularly useful in cases where there are members of a gene family that have been mis-identified as being members of a single cluster. Subclustering permits the sequences of such members to be viewed independently for the selection of probes that will detect the expression of such members apart from other members of the same family.

Probes for use as normalization controls can be selected and attached to microarrays of the invention as known in the art.

Q-PCR Related Embodiments of the Invention

In a second aspect, the invention provides Q-PCR based methods for detecting expressed sequences in a biological sample. An expressed sequence can be any of those in a transcriptome and thus can be any transcribed sequence. In one embodiment, the invention provides for the use of quantitative reverse transcription PCR (RT-PCR) based assay methods for the detection of expressed sequences in a biological sample containing RNA transcripts. In RT-PCR, a starting RNA template, such as mRNA, is first converted to DNA by use of a reverse transcriptase activity. The quantitative RT-PCR based methods may also be used with RNA transcripts produced by in vitro transcription (IVT) of cDNA produced from RNA transcripts of a biological sample. The cDNA may be of a particular transcript of interest or of an “in toto” or “global” conversion of transcribed RNAs. The Q-PCR based methods may also be used with the cDNAs per se as well as with a particular mRNA or cDNA species. The methods may also be used with amplified RNA (aRNA) or the corresponding cDNA thereof, as the starting template. Primers and probes for detecting expressed sequences and articles of manufacture such as kits containing such primers and probes are provided by the invention.

The design and selection of primers and optional probes for Q-PCR can be made by review of sequences at the 3′ region of cellular transcripts, which can be identified by various means, including experimentally or by selection based upon sequences from publicly available sources, optionally supplemented, as described above. As noted, the use of EST sequences may be preceded by analyzing them for untrimmed, low-quality sequence information, correct orientation, false priming, false clustering, and alternative splicing followed by correction or removal of sequences from consideration as known in the art. EST sequences may also be analyzed for alternative polyadenylation to confirm the existence of, and identify the location of, more than one polyadenylation site.

As a non-limiting example, amplification of the 3′ region of the human beta actin sequence may be performed as described herein. This sequence has been found to be expressed at relatively consistent levels in both cancer and non-cancer breast cells and as such may be used as a reference sequence as disclosed herein. A PCR amplicon of 92 basepairs that is within 20 nucleotides of the polyadenylation site may be used to detect expression of the human beta actin sequence as described in the Examples below.

As further non-limiting examples, amplification of the 3′ region of the human “ubiquitin C” sequence; the human succinate dehydrogenase complex, subunit A flavoprotein sequence; or the human ribosomal protein L13a (RPL13A) may be used as a reference sequence as described herein. While the amplification and detection of such sequences may be via any Q-PCR based method described herein, preferred embodiments include the use of nucleic acid binding dyes such as, but not limited to, Sybr Green.

The primers and optional probes may also be selected after analysis of sequence clusters as described above. Such analysis may be used to design or select primer or probe sequences that are capable of detecting one of a family of related sequences, optionally by use of the same Q-PCR primer pair. As a non-limiting example, two closely related transcribed sequences with similar or nearly identical sequences at the 3′ region may be simultaneously amplified by Q-PCR using a single primer pair that amplifies all or part of the 3′ region of both transcribed sequences, and with use of a probe sequence complementary to a unique portion of the amplified region of one of the two transcribed sequences, may be used to detect the expression of one transcribed sequence and not the other. Of course this can also be conducted with the use of a primer pair that is unique to the probe being used.

Alternatively, the invention may be performed in “multiplex” mode such that in the above non-limiting examples, differentially labeled Q-PCR probes that specifically hybridize to each of the two transcribed sequences (for a total of two probes) may be used to permit detection of each of the two transcribed sequences simultaneously by detection of the two different labels. As noted herein, the invention may be practiced based upon a probe hydrolysis method or other Q-PCR method. This includes the use of methods comprising a labeled probe that forms a hairpin structure to permit FRET.

Primers that amplify at the 3′ region of transcribed sequences can be designed by first identifying homology or consensus sequences within a portion of the 3′ region based upon an alignment of more than one sequence; identifying potential primer and probe sequences, such as those with a higher GC (guanine and cytosine) content or that are likely to have a particular melting temperature (Tₘ,) within the homologous regions; and selecting particular sequences for use as forward and reverse primers as well as probes. In the case of RT-PCR, the selection of primer sequences may also include consideration of the primer used for the reverse transcription step. The selection of primer and probe sequences may be performed with the aid of a computer program such as those available on the internet as NetPrimer and HyTher. Other possibilities include OLIGO from Molecular Biology Insights Inc., Cascade, Colo. Important features when designing oligonucleotides to be used as amplification primers include, but are not limited to, an appropriate size amplification product to facilitate detection (e.g., by electrophoresis), similar melting temperatures for the members of a pair of primers, and the length of each primer (i.e., the primers need to be long enough to anneal with sequence-specificity and to initiate synthesis but not so long that fidelity is reduced during oligonucleotide synthesis). Typically, oligonucleotide primers are about 6 to about 30 nucleotides in length (e.g., about 8, about 10, about 12, about 14, about 16, about 18, about 20, about 22, about 24, about 26, about 28, or about 30 nucleotides in length).

The primers may be designed to amplify a region (or amplicon) of any reasonable length over the lengths of the primers themselves. Therefore, amplicons of about 40 nucleotides, about 50 nucleotides, about 60 nucleotides, about 70 nucleotides, about 80 nucleotides, about 90 nucleotides, about 100 nucleotides, about 120 nucleotides, about 140 nucleotides, about 160 nucleotides, about 180 nucleotides, about 200 nucleotides, about 225 nucleotides, about 250 nucleotides, or more than any of these values may be practiced in accord with the instant invention. Preferred amplicons are less than about 200 nucleotides or less than about 100 nucleotides to permit rapid analysis during Q-PCR.

Designing oligonucleotides to be used as Q-PCR probes can be performed in a manner similar to the design of primers, although the separation between donor and quencher/acceptor moieties in a single probe must not be so great as to prevent fluorescent resonance energy transfer (FRET). In the case of two members of a pair of probes (one containing a donor and one containing a quencher or acceptor moiety), they are preferably designed to anneal to an amplification product within no more than 5 nucleotides of each other (e.g., within no more than 1, 2, 3, or 4 nucleotides of each other) on the same strand such that fluorescent resonance energy transfer (FRET) can occur. It is to be understood, however, that longer separation distances (such as 6 or more nucleotides) are possible if the moieties are appropriately positioned relative to each other (such as by use of a linker) such that FRET can occur. In addition, probes can be designed to hybridize to targets that contain a mutation or polymorphism, thereby allowing differential detection of transcribed sequences based on either absolute hybridization of different probes or optionally via differential melting temperatures between, for example, each probe and each amplification product corresponding to a transcribed sequence to be distinguished. In some embodiments of the invention, the 3′ ends of the probes are blocked to prevent their utilization to primer nucleic acid synthesis. Non-limiting examples of blocking groups include PO₄, NH₂ or a blocked base.

Conventional PCR techniques are disclosed in U.S. Pat. Nos. 4,683,202, 4,683,195, 4,800,159, and 4,965,188. Briefly, PCR typically employs two oligonucleotide primers that bind to a selected nucleic acid template (e.g., DNA or RNA) and its complement. Primers for use in the present invention include oligonucleotides capable of serving as the start of nucleic acid synthesis within the 3′ region of a transcribed nucleic acid sequence. The nucleic acid synthesis is usually mediated by a thermostable polymerase activity. A primer may be produced synthetically via a DNA synthesizer. A primer is preferably single-stranded for maximum efficiency in amplification, but a primer may also be used after denaturation, such as by heating, to separate the two strands.

The term “thermostable polymerase” refers to a polymerase enzyme that is heat stable and thus does not irreversibly denature when subjected to the elevated temperatures for the time necessary to effect denaturation of double-stranded template nucleic acids. The polymerase activity catalyzes the formation of primer extension products complementary to a template while a 5′ to 3′ exonuclease activity may also be present. Generally, nucleic acid synthesis is initiated at the 3′ end of each primer and proceeds in the 5′ to 3′ direction along the template strand. Thermostable polymerases isolated from many organisms may be used in the practice of the invention. Polymerases that are not thermostable also can be employed in PCR if they are replenished during PCR.

PCR assays can be used with unpurified nucleic acid templates or where the template may be a minor fraction of a complex mixture, such as, but not limited to, mRNAs from tissues or cells. Such tissues or cells may be those of a biological sample. As a non-limiting example, the mRNA template is combined with the oligonucleotide primers and with other PCR reagents under reaction conditions suitable for primer extension. Conditions suitable for chain extension reactions are known in the art. They generally include an appropriate buffer, MgCl₂, template, oligonucleotide primers, thermostable polymerase activity (and reverse transcriptase activity in the case of an RNA template), and the necessary nucleotides or analogs thereof.

The newly synthesized strands form a double-stranded molecule that can be used in the succeeding steps of the reaction. The steps of strand separation, annealing, and elongation can be repeated as often as needed to produce a quantity of amplification products corresponding to the target sequence present in an expressed nucleic acid molecule. The limiting factors in the reaction are usually the amounts of primers, thermostable enzyme, and nucleoside triphosphates present in the reaction. The cycling steps (i.e., amplification and hybridization) are preferably repeated at least once. The number of cycling steps will depend on a variety of factors, including the nature of the sample. As a non-limiting example, if the sample is a complex mixture of nucleic acids, more cycling steps may be required to amplify the target sequence sufficient for detection. Generally, the cycling steps are repeated at least about 10 or about 20 times, but may be repeated as many as about 40 or more, about 60 or more, or even about 100 or more times.

FRET technology is discussed in U.S. Pat. Nos. 4,996,143, 5,565,322, 5,849,489, and 6,162,603. FRET is based on the fact that when a donor and a corresponding acceptor moiety are positioned within a certain distance of each other, energy transfer takes place between the two moieties. The transferred can be visualized or otherwise detected and/or quantitated. Alternatively, the transfer can be a quenching of the fluorescence of the donor such that interruption of the transfer results in the emission of detectable fluorescence.

As used herein with respect to donor and corresponding quencher or acceptor moieties, “corresponding” refers to a quencher or acceptor moiety having an emission spectrum that overlaps the excitation spectrum of the donor fluorescent moiety. The wavelength maximum of the emission spectrum of the quencher or acceptor moiety preferably should be at least 100 nm greater than the wavelength maximum of the excitation spectrum of the donor fluorescent moiety. This results in efficient non-radiative energy transfer between the two moieties.

Fluorescent donor and corresponding quencher or acceptor moieties are generally chosen for (a) high efficiency Forster energy transfer; (b) a large final Stokes shift (>100 nm); (c) shift of the emission as far as possible into the red portion of the visible spectrum (>600 nm); and (d) shift of the emission to a higher wavelength than the Raman water fluorescent emission produced by excitation at the donor excitation wavelength. For example, a donor fluorescent moiety can be chosen that has its excitation maximum near a laser line (for example, Helium-Cadmium 442 nm or Argon 488 nm), a high extinction coefficient, a high quantum yield, and a good overlap of its fluorescent emission with the excitation spectrum of the corresponding quencher or acceptor moiety. A corresponding quencher or acceptor moiety can be chosen that has a high extinction coefficient, a high quantum yield, a good overlap of its excitation with the emission of the donor fluorescent moiety, and emission in the red part of the visible spectrum (>600 nm).

Representative donor fluorescent moieties that can be used with various acceptor fluorescent moieties in FRET technology include fluorescein, Lucifer Yellow, B-pliycoerythrin, 9-acridineisothiocyanate, Lucifer Yellow VS, 4-acetamido-4′-isothiocyanatostilbene-2,2′-disulfonic acid, 7-diethylamino-3-(4′-isothiocyanatophenyl)-4-methylcoumarin, succinimidyl 1-pyrenebutyrate, and 4-acetamido-4′-isothiocyanatostilbene-2,2′-disulfonic acid derivatives. Representative acceptor fluorescent moieties, depending upon the donor fluorescent moiety used, include LC™-RED 640 (LightCycler™-Red 640-N-hydroxysuccinimide ester), LC™-RED 705 (LightCycler™-Red 705-Phosphoramidite), cyanine dyes such as CY5 and CY5.5, Lissamine rhodamine B sulfonyl chloride, tetramethyl rhodamine isothiocyanate, rhodamine x isothiocyanate, erythrosine isothiocyanate, fluorescein, diethylenetriamine pentaacetate or other chelates of Lanthanide ions (e.g., Europium, or Terbium). Donor and acceptor fluorescent moieties can be obtained, for example, from Molecular Probes (Junction City, Oreg.) or Sigma Chemical Co. (St. Louis, Mo.).

The donor and quencher or acceptor moieties can be attached to the appropriate probe oligonucleotide via a linker. The length of each linker arm can be important, as the linker arms will affect the distance between the donor and the quencher or acceptor moieties. The length of a linker for the purpose of the present invention is the distance in Angstroms (Å) from the nucleotide base to the fluorescent moiety. In general, a linker is from about 10 to about 25 Å. A variety of linkers are known in the field and may be used in the present invention.

The invention provides methods for detecting the presence or absence of an expressed sequence in a biological sample from an individual. The methods include performing at least one cycling step that includes amplifying and hybridizing where the amplification step includes contacting the biological sample with a pair of Q-PCR primers to produce a Q-PCR amplification product if the expressed sequence to be amplified is present in the sample. Each of the primers anneals to a target within (or adjacent to in cases where a primer anneals to all or part of the poly A tail) a nucleic acid sequence to be amplified such that at least a portion of the amplification product contains nucleic acid sequence from the 3′ region of the sequence. More importantly, the amplification product contains the nucleic acid sequences that are complementary to one or more Q-PCR probes. A hybridizing step includes contacting the sample with one or more Q-PCR probes. Multiple cycling steps can be performed, preferably in a thermocycler.

PCR amplification synthesizes nucleic acid molecules that are complementary to one or both strands of a template nucleic acid. Amplifying a nucleic acid molecule typically includes denaturing the template nucleic acid, annealing primers to the template nucleic acid at a temperature that is below the melting temperatures of the primers, and enzymatically elongating from the primers to generate an amplification product. The denaturing, annealing and elongating steps each can be performed once per cycle. Generally, however, the denaturing, annealing and elongating steps are performed in multiple cycles such that the amount of amplification product is increasing, often times exponentially, although exponential amplification is not required by the present methods. Amplification typically requires the presence of deoxyribonucleoside triphosphates, a DNA (thermostable) polymerase enzyme and an appropriate buffer and/or co-factors for optimal activity of the polymerase enzyme.

If amplification of an expressed nucleic acid occurs and an amplification product is produced, the step of hybridizing results in the annealing of one or more probe molecules to the product via base pair complementarity. Hybridization conditions typically include a temperature that is below the melting temperature of the probes from the amplification product but that avoids non-specific hybridization of the probes.

In the case of probe hydrolysis to generate a detectable signal, the 5′ to 3′ exonuclease activity of a (thermostable) DNA polymerase is used to release a fluorescent moiety from being quenched or subdued by a quencher or acceptor present on the same probe molecule.

In the case of a pair of probes, each containing one of a donor and quencher or acceptor moieties, the presence of FRET indicates the presence of a transcribed sequence in the biological sample, and the absence of FRET indicates the absence of a transcribed sequence in the biological sample.

Within each thermocycler run, control samples can be cycled as well. Positive control samples can amplify control nucleic acid template (preferably one other than the transcribed sequence to be detected) using, as a non-limiting example, control primers and control probes. Positive control samples can also amplify, as a non-limiting example, a plasmid construct containing the transcribed nucleic acid sequence. Such a plasmid control can be amplified internally (such as within each biological sample) or in separate samples run side-by-side with the test samples. Each thermocycler run also should include a negative control that, for example, lacks template nucleic acid. Such controls are indicators of the success or failure of the amplification, hybridization, and/or detection steps. Therefore, control reactions can readily determine, for example, the ability of primers to anneal with sequence-specificity and to initiate elongation, as well as the ability of probes to hybridize with sequence-specificity.

As noted herein, a common FRET technology format utilizes TAQMAN® technology to detect the presence or absence of an amplification product, and hence, the presence or absence of a transcribed sequence. The technology utilizes one single-stranded hybridization probe labeled with two moieties. When a first fluorescent moiety is excited with light of a suitable wavelength, the absorbed energy is transferred to a second quencher or acceptor moiety according to the principles of FRET. The second fluorescent moiety is preferably a quencher molecule. During the annealing step of the PCR reaction, the labeled hybridization probe binds to the target DNA (i.e., the amplification product) and is degraded by the 5′ to 3′ exonuclease activity of the Taq Polymerase during the subsequent elongation phase. After release, the excited fluorescent moiety and the quencher moiety become spatially separated from one another such that the emission from the first fluorescent moiety can be detected.

Another FRET technology format utilizes two hybridization probes. Each probe can be labeled with a different fluorescent moiety and the two probes are generally designed to hybridize in close proximity to each other in a target DNA molecule such as an amplification product. Efficient FRET can only take place when the fluorescent moieties are in direct local proximity (for example, within 5 nucleotides of each other as described herein) and when the emission spectrum of the donor fluorescent moiety overlaps with the absorption spectrum of the acceptor fluorescent moiety. The intensity of the emitted signal can be correlated with the number of original target DNA molecules (e.g., the number of transcription products in a starting sample).

Yet another FRET technology format utilizes molecular beacon technology to detect the presence or absence of an amplification product, and hence, the presence or absence of a transcribed sequence. Molecular beacon technology uses a hybridization probe labeled with a donor fluorescent moiety and an acceptor fluorescent moiety. The acceptor fluorescent moiety is generally a quencher, and the fluorescent labels are typically located at each end of the probe. Molecular beacon technology uses a probe oligonucleotide having sequences that permit secondary structure formation (e.g., a hairpin). As a result of secondary structure formation within the probe, both fluorescent moieties are in spatial proximity when the probe is in solution. After hybridization to the target nucleic acids (i.e., the amplification products), the secondary structure of the probe is disrupted and the fluorescent moieties become separated from one another such that after excitation with light of a suitable wavelength, the emission of the first fluorescent moiety can be detected.

As an alternative to detection using FRET technology, an amplification product can be detected using a nucleic acid binding dye such as a fluorescent DNA binding dye. After interaction with the double-stranded nucleic acid, the nucleic acid bound dyes emit a fluorescence signal after excitation with light at a suitable wavelength. A nucleic acid intercalating dye may also be used. When nucleic acid binding dyes are used, a melting curve analysis is usually performed for confirmation of the presence of the amplification product.

Detection of Gene Expression

In specific non-limiting embodiments, the present invention provides methods useful for detecting cancer cells, facilitating diagnosis of cancer and the severity of a cancer (e.g., tumor grade, tumor burden, and the like) in a subject, facilitating a determination of the prognosis of a subject, and assessing the responsiveness of the subject to therapy (e.g., by providing a measure of therapeutic effect through, for example, assessing tumor burden during or following a chemotherapeutic regimen). Preferably, the methods are used in relation to human subjects and are directed to neoplasms and cancers, including but not limited to gene expression in cells from sarcomas, carcinomas, lymphomas, leukemias, biopsies, neuroendocrine carcinomas, sarcomas of the urinary bladder, metastatic carcinomas (such as but not limited to from the prostate, colon-rectum, uterine, cervix, and endometrium), malignant lymphomas (such as but not limited to Hodgkins, non-Hodgkins B cell, non-Hodgkins T cell), mengiomas, and/or renal cell carcinomas. Other cancers include those of the adrenal glands, such as but not limited to Pheochromocytoma and Neuroblastoma; of the bladder, such as but not limited to Papillary and/or Transitional cancers or tumors; of the bone, such as but not limited to Osteosarcoma, Chondrosarcoma, and Ewings Sarcoma; of the brain, such as but not limited to astrocytoma and oligodendroglioma; of the breast, such as but not limited to Invasive Ductal Carcinoma, Lobular Carinoma, and mucinous/medullary/tubular cancers or tumors; of the cervix, such as but not limited to Squamous Cell Carcinoma and Adencarcinoma; of the Small Intestine, such as but not limited to Adenocarcinoma of Small Intestine and Carcinoid Tumor; of the Colon/Large Intestine, such as but not limited to Adenocarcinoma of Large Intestine and Carcinoid Tumor (neuroendocrine origin); of the Rectum, such as but not limited to Squamous Cell Carcinoma; of the Esophagus, such as but not limited to Esophageal Adenocarcinoma, Esophageal Squamous Cell Carcinoma, and Barrett's Esophagus; of the Gall Bladder, such as but not limited to Gall Bladder Adenocarcinoma and Bile Duct Adenocarcinoma; of the Kidney, such as but not limited to Renal Cell Carcinoma; of the Larynx, such as but not limited to Squamous Cell Carcinoma; of the Liver, such as but not limited to Hepatocellular Carcinoma and Cholangiocarcinoma; of the Lung, such as but not limited to Adenocarcinoma, Squamous Cell Carcinoma, Large Cell Carcinoma, Small Cell Carcinoma, and Mesothelioma; of the Ovary, such as but not limited to Serous Carcinoma, Mucinous Carcinoma, Clear Cell Carcinoma, and Germ Cell Tumors; of the Pancreas, such as but not limited to Pancreatic Carcinoma; of the Prostate, such as but not limited to Prostate carcinoma; of the Skin, such as but not limited to Squamous Cell Carcinoma, Basal Cell Carcinoima, and Melanoma; of Soft Tissue, such as but not limited to Rhabdomyosarcoma, Synovial Sarcoma, Fibrosarcoma, liposarcoma, and mfh (malignant fibros histocytoma); of the Stomach, such as but not limited to Adenocarcinoma and Gastrointestinal Stromal Tumor; of the Testes, such as but not limited to Germ Cell Tumors, Embryonal carcinoma, and Seminoma; of the Thyroid, such as but not limited to Papillary Carcinoma and follicular carcinoma and/or medullary carcinoma; and of the Uterus, such as but not limited to Leiomyosarcoma and Endometrial Adenocarcinoma.

The present invention also provides methods for differentiating the above from nephrogenic adenoma, cellular changes in gene expression due to topical chemotherapy (e.g. treatment with thiotepa, mitomycin, or Bacillus Calmette-Guerin (BCG) vaccine), cellular changes in gene expression due to systemic chemotherapy (e.g. cyclophosphamide), radiation induced changes in cellular gene expression, and/or virus induced changes in cellular gene expression (e.g. infection by human polyomavirus) by differential gene expression analysis using microarrays or Q-PCR. The last of these is particularly important to differentiate from high grade transitional cell carcinoma.

Cell containing samples of the above may be isolated from a subject for preparation of polynucleotides for hybridization to a microarray of the invention or for Q-PCR based analysis as described herein. Non-limiting examples of such samples include biopsy samples and cytological specimens that are either spontaneous or abraded exfoliates, such as fine needle aspirates obtained via a biopsy procedure. Particularly preferred are specimens collected via a PAP smear, ductal lavage, fine needle aspiration, drawing blood or plasma or serum, prostate massage, sputum (including saliva, bronchial brush or bronchial wash), stool, semen, urine, or other bodily fluid (including ascitic fluid, cerebral spinal fluid (CSF), bladder wash, pleural fluid, and the like). Non-limiting examples of tissues susceptible to fine needle aspiration include lymph node, lung, thyroid, breast, and liver.

Detection can be based on determination of one or more polynucleotides as differentially expressed in a cell or tissue sample by use of a microarray of the invention. Such a microarray may comprise probes capable of hybridizing to, and thus detecting, sequences expressed in the cell or tissue sample. The transcripts expressed by a cell or tissue may be directly hybridized to the microarray in a detectable manner, such as, but not limited to, labeling the polynucleotides prior to hybridization. Alternatively, the expressed transcripts may be converted into cDNA molecules or amplified to produce DNA or RNA molecules that are hybridized to the microarray in a detectable manner. The converted or amplified molecules are preferably labeled prior to hybridization to the microarray.

Alternatively, analysis of gene expression in a cell or tissue sample may be performed by use of Q-PCR based amplification of the 3′ region of one or more expressed sequences of interest. Such analysis may comprise the use of primers and optional probes complementary to the 3′ region of an expressed sequence to permit amplification thereof as described herein. The sequences expressed in a cell or tissue may be directly amplified, such as by reverse transcription PCR (RT-PCR) coupled with Q-PCR, or may first be converted to cDNA before Q-PCR. The cDNA may also be used to produce amplified RNA molecules that are analyzed by RT-PCR coupled with Q-PCR. The Q-PCR amplified molecules may be optionally labeled to facilitate their detection as desired.

In one embodiment of the invention, the microarrays of the invention are hybridized to polynucleotides obtained from a sample is one that has been formalin fixed and paraffin embedded (also referred to as an FFPE sample). Pending U.S. patent application Ser. No. 10/329,282, filed Dec. 23, 2002, which is hereby incorporated by reference as if fully set forth, describes the amplification of expressed nucleic acids from an FFPE sample. Such amplified nucleic acids may be hybridized to a microarray of the invention for diagnostic purposes or to correlate the transcriptome of cells of an FFPE sample with the disease, disease state, disease outcome, or disease response to treatment(s), of the subject from whom the sample was obtained.

In another embodiment of the invention, nucleic acids from an FFPE sample, optionally amplified as described in the above paragraph, are analyzed by Q-PCR as described herein. The Q-PCR based analysis can be used for diagnostic purposes, such as by detection of an expressed sequence as over or underexpressed in a manner that corresponds with a disease, disease state, disease outcome, or disease response to treatment(s) of the subject from whom the sample was obtained.

In all of the above, the samples are optionally microdissected to isolate cells of interest for the preparation and isolation of polynucleotides for hybridization to a microarray of the invention or for analysis by Q-PCR as described herein.

As noted above, the microarrays of the invention may be hybridized to polynucleotides as well as amplified polynucleotides corresponding to expressed gene sequences. The polynucleotides hybridized to a microarray of the invention may be labeled to facilitate their detection after hybridization to a microarray. Detecting labeled polynucleotides can be conducted by standard methods used to detect the labeled sequences. For example, fluorescent labels or radiolabels can be detected directly. Other labeling techniques may require that a label such as biotin or digoxigenin be incorporated into the DNA or RNA during amplification of and detected by an antibody or other binding molecule (e.g. streptavidin) that is either labeled or which can bind a labeled molecule itself. For example, a labeled molecule can be an anti-streptavidin antibody or anti-digoxigenin antibody conjugated to either a fluorescent molecule (e.g. fluorescein isothiocyanate, Texas red and rhodamine), or an enzymatically active molecule. Whatever the label on the newly synthesized molecules, and whether the label is directly in the DNA or conjugated to a molecule that binds the DNA (or binds a molecule that binds the DNA), the labels (e.g. fluorescent, enzymatic, chemiluminescent, or calorimetric) can be detected by a laser scanner or a CCD camera, or X-ray film, depending on the label, or other appropriate means for detecting a particular label.

An amplified target polynucleotide can be detected on a microarray by virtue of labeled-nucleotides (e.g. dNTP-fluorescent label for direct labeling; and dNTP-biotin or dNTP-digoxigenin for indirect labeling) incorporated during amplification. For indirectly labeled DNA, the detection is carried out by fluorescence or other enzyme conjugated streptavidin or anti-digoxigenin antibodies. The method employs detection of the polynucleotides by detecting incorporated label in the newly synthesized complements to the polynucleotide targets. For this purpose, any label that can be incorporated into DNA as it is synthesized can be used, e.g. fluoro-dNTP, biotin-dNTP, or digoxigenin-dNTP, as described above and are known in the art. In a differential expression system, amplification products derived from different biological sources can be detected by differentially (e.g., red dye and green dye) labeling the amplified target polynucleotides based on their origins.

In a preferred embodiment, amplified RNA, such as that produced by the methods described in U.S. patent application Ser. No. 10/062,857, filed Oct. 25, 2001, carry the labels. The anchor or oligo-dT portions of the primers used to amplify RNA generally have labels incorporated during their use in nucleic acid synthesis. The promoter regions of the promoter-primer oligonucleotides may also include direct or indirectly detectable labels as long as incorporations of the labels do not significantly hamper their functionality as promoters for the corresponding RNA polymerases.

For detection, light detectable means are preferred, although other methods of detection may be employed, such as radioactivity, atomic spectrum, and the like. For light detectable means, one may use fluorescence, phosphorescence, absorption, chemiluminescence, or the like. One of the most convenient means is fluorescence, which may take many forms. One may use individual fluorescers or pairs of fluorescers, particularly where one wishes to have a plurality of emission wavelengths with large Stokes shifts (at least 20 nm). Illustrative fluorescers include fluorescein, rhodamine, Texas red, cyanine dyes, phycoerythrins, thiazole orange and blue, etc. When using pairs of dyes, one may have one dye on one molecule and the other dye on another molecule which binds to the first molecule. The important factor is that the two dyes when the two components are bound are close enough for efficient energy transfer.

Another way of labeling which may find use in the subject invention is isotopic labeling, in which one or more of the nucleotides is labeled with a radioactive label, such as ³²S, ³²P, ³H, or the like. Another means of labeling is fluorescent labeling in which a fluorescently tagged nucleotide, e.g. CTP, is incorporated into the polynucleotide (e.g. amplified RNA) product during transcription. Fluorescent moieties which may be used to tag nucleotides for producing labeled antisense RNA include: fluorescein, the cyanine dyes, such as Cy3, Cy5, Alexa 542, Bodipy 630/650, and the like. Particularly preferred in the practice of the invention is the use of Cy3 or Cy5 with the use of a generic mRNA control that is labeled with the other of Cy3 and Cy5.

Kits and Articles of Manufacture

The invention also provides articles of manufacture such as kits for the practice of Q-PCR based methods of the invention. The article of manufacture or kit preferably contains a reagent set comprising buffers, primers and probe and enzymes ready to load into one or more reaction tubes along with extracted or amplified RNA samples, as a non-limiting example. The sequences of the primers and probes are preferably complementary to the 3′ region of one or more cellular transcripts and capable of quantitatively amplifying sequences within the 3′ region as described herein. In one embodiment, the Q-PCR reaction reagents for amplification of a particular sequence are provided in a single tube to which nucleic acid material for amplification and optional enzymatic reagents are added to reduce the potential for contamination, simplify the handling of reagents, and decrease the likelihood of error. The tube preferably contains a frozen mixture, optionally with controls, in a pre-determined total reaction volume.

A kit according to the present invention also preferably comprises suitable packaging material. Preferably, the packaging includes a label or instructions for the use of the article in a method disclosed herein.

Having now generally described the invention, the same will be more readily understood through reference to the following example which is provided by way of illustration, and is not intended to be limiting of the present invention, unless specified.

### EXAMPLES

### Example 1

The human beta actin sequence is expressed in many cell types. The sequence has been deposited with GenBank and identified with accession number X00351 or version X00351.1 (as well as J00074, M10278, and GI:28251). The deposited sequence is 1761 nucleotides long and is as follows:

| | |
|---|---|
| 1 | ttgccgatcc gccgcccgtc cacacccgcc gccagctcac catggatgat gatatcgccg |
| 61 | cgctcgtcgt cgacaacggc tccggcatgt gcaaggccgg cttcgcgggc gacgatgccc |
| 121 | cccgggccgt cttcccctcc atcgtggggc gccccaggca ccagggcgtg atggtgggca |
| 181 | tgggtcagaa ggattcctat gtgggcgacg aggcccagag caagagaggc atcctcaccc |
| 241 | tgaagtaccc catcgagcac ggcatcgtca ccaactggga cgacatggag aaaatctggc |
| 301 | accacacctt ctacaatgag ctgcgtgtgg ctcccgagga gcaccccgtg ctgctgaccg |
| 361 | aggcccccct gaaccccaag gccaaccgcg agaagatgac ccagatcatg tttgagacct |
| 421 | tcaacacccc agccatgtac gttgctatcc aggctgtgct atccctgtac gcctctggcc |
| 481 | gtaccactgg catcgtgatg gactccggtg acggggtcac ccacactgtg cccatctacg |
| 541 | aggggtatgc cctcccccat gccatcctgc gtctggacct ggctggccgg gacctgactg |
| 601 | actacctcat gaagatcctc accgagcgcg gctacagctt caccaccacg gccgagcggg |
| 661 | aaatcgtgcg tgacattaag gagaagctgt gctacgtcgc cctggacttc gagcaagaga |
| 721 | tggccacggc tgcttccagc tcctccctgg agaagagcta cgagctgcct gacggccagg |
| 781 | tcatcaccat tggcaatgag cggttccgct gccctgaggc actcttccag ccttccttcc |
| 841 | tgggcatgga gtcctgtggc atccacgaaa ctaccttcaa ctccatcatg aagtgtgacg |
| 901 | tggacatccg caaagacctg tacgccaaca cagtgctgtc tggcggcacc accatgtacc |
| 961 | ctggcattgc cgacaggatg cagaaggaga tcactgccct ggcacccagc acaatgaaga |
| 1021 | tcaagatcat tgctcctcct gagcgcaagt actccgtgtg gatcggcggc tccatcctgg |
| 1081 | cctcgctgtc caccttccag cagatgtgga tcagcaagca ggagtatgac gagtccggcc |
| 1141 | cctccatcgt ccaccgcaaa tgcttctagg cggactatga cttagttgcg ttacaccctt |
| 1201 | tcttgacaaa acctaacttg cgcagaaaac aagatgagat tggcatggct ttatttgttt |
| 1261 | tttttgtttt gttttggttt tttttttttt tttggcttga ctcaggattt aaaaactgga |
| 1321 | acggtgaagg tgacagcagt cggttggagc gagcatcccc caaagttcac aatgtggccg |
| 1381 | aggactttga ttgcacattg ttgttttttt aatagtcatt ccaaatatga gatgcattgt |
| 1441 | tacaggaagt cccttgccat cctaaaagcc accccacttc tctctaagga gaatggccca |
| 1501 | gtcctctccc aagtccacac aggggaggtg atagcattgc tttcgtgtaa attatgtaat |
| 1561 | gcaaaatttt tttaatcttc gccttaatac ttttttattt tgttttattt tgaatgatga |
| 1621 | gccttcgtgc ccccccttcc ccctttttgt cccccaactt gagatgtatg aaggcttttg |
| 1681 | gtctccctgg gagtgggtgg aggcagccag ggcttacctg tacactgact tgagaccagt |
| 1741 | tgaataaaag tgcacacctt a |

Position 1761 is identified as the polyadenylation site, and the underlined portion above is a 92 nucleotide long amplicon that is practiced in accordance with the instant invention. The amplicon spans nucleotides 1650 to 1741 and is amplified by a forward Q-PCR primer from position 1650 to 1683 (34 nucleotides in length) and a reverse Q-PCR primer complementary to positions 1741 to 1717 (25 nucleotides in length).

This example is exemplary of situations where the sequence to be detected is within a region less than about 150 nucleotides from the site of polyadenylation. Indeed, this example has the detected sequence within less than about 110 nucleotides from the site of polyadenylation.

### Example 2

The human sequence referred to as “similar to ubiquitin C, clone MGC:8448 IMAGE:2821375” is expressed in many cell types. The sequence has been deposited with GenBank and identified with accession number BC000449 or version BC000449.1 (as well as GI:12653358). This deposited sequence is 2210 nucleotides long and is as follows:

| | |
|---|---|
| 1 | ggcacgaggc gggatttggg tcgcggttct tgtttgtgga tcgctgtgat cgtcacttga |
| 61 | caatgcagat cttcgtgaag actctgactg gtaagaccat caccctcgag gttgagccca |
| 121 | gtgacaccat cgagaatgtc aaggcaaaga tccaagataa ggaaggcatc cctcctgacc |
| 181 | agcagaggct gatctttgct ggaaaacagc tggaagatgg gcgcaccctg tctgactaca |
| 241 | acatccagaa agagtccacc ctgcacctgg tgctccgtct cagaggtggg atgcaaatct |
| 301 | tcgtgaagac actcactggc aagaccatca cccttgaggt ggagcccagt gacaccatcg |
| 361 | agaacgtcaa agcaaagatc caggacaagg aaggcattcc tcctgaccag cagaggttga |
| 421 | tctttgccgg aaagcagctg gaagatgggc gcaccctgtc tgactacaac atccagaaag |
| 481 | agtctaccct gcacctggtg ctccgtctca gaggtgggat gcagatcttc gtgaagaccc |
| 541 | tgactggtaa gaccatcacc ctcgaggtgg agcccagtga caccatcgag aatgtcaagg |
| 601 | caaagatcca agataaggaa ggcattcctc ctgatcagca gaggttgatc tttgccggaa |
| 661 | aacagctgga agatggtcgt accctgtctg actacaacat ccagaaagag tccaccttgc |
| 721 | acctggtact ccgtctcaga ggtgggatgc aaatcttcgt gaagacactc actggcaaga |
| 781 | ccatcaccct tgaggtcgag cccagtgaca ctatcgagaa cgtcaaagca aagatccaag |
| 841 | acaaggaagg cattcctcct gaccagcaga ggttgatctt tgccggaaag cagctggaag |
| 901 | atgggcgcac cctgtctgac tacaacatcc agaaagagtc taccctgcac ctggtgctcc |
| 961 | gtctcagagg tgggatgcag atcttcgtga agaccctgac tggtaagacc atcaccctcg |
| 1021 | aagtggagcc gagtgacacc attgagaatg tcaaggcaaa gatccaagac aaggaaggca |
| 1081 | tccctcctga ccagcagagg ttgatctttg ccggaaaaca gctggaagat ggtcgtaccc |
| 1141 | tgtctgacta caacatccag aaagagtcca ccttgcacct ggtgctccgt ctcagaggtg |
| 1201 | ggatgcagat cttcgtgaag accctgactg gtaagaccat cactctcgag gtggagccga |
| 1261 | gtgacaccat tgagaatgtc aaggcaaaga tccaagacaa ggaaggcatc cctcctgatc |
| 1321 | agcagaggtt gatctttgct gggaaacagc tggaagatgg acgcaccctg tctgactaca |
| 1381 | acatccagaa agagtccacc ctgcacctgg tgctccgtct tagaggtggg atgcagatct |
| 1441 | tcgtgaagac cctgactggt aagaccatca ctctcgaagt ggagccgagt gacaccattg |
| 1501 | agaatgtcaa ggcaaagatc caagacaagg aaggcatccc tcctgaccag cagaggttga |
| 1561 | tctttgctgg gaaacagctg gaagatggac gcaccctgtc tgactacaac atccagaaag |
| 1621 | agtccaccct gcacctggtg ctccgtctta gaggtgggat gcagatcttc gtgaagaccc |
| 1681 | tgactggtaa gaccatcact ctcgaagtgg agccgagtga caccattgag aatgtcaagg |
| 1741 | caaagatcca agacaaggaa ggcatccctc ctgaccagca gaggttgatc tttgctggga |
| 1801 | aacagctgga agatggacgc accctgtctg actacaacat ccagaaagag tccaccctgc |
| 1861 | acctggtgct ccgtctcaga ggtgggatgc agatcttcgt gaagaccctg actggtaaga |
| 1921 | ccatcaccct cgaggtggag cccagtgaca ccatcgagaa tgtcaaggca aagatccaag |
| 1981 | ataaggaagg catccctcct gatcagcaga ggttgatctt tgctgggaaa cagctggaag |
| 2041 | atggacgcac cctgtctgac tacaacatcc agaaagagtc cactctgcac ttggtcctgc |
| 2101 | gcttgagggg gggtgtctaa gtttcccctt ttaaggtttc aacaaatttc attgcacttt |
| 2161 | cctttcaata aagttgttgc attcccaaaa aaaaaaaaaa aaaaaaaaaa |

This deposited sequence was replaced by a newer sequence referred to as “Homo sapiens ubiquitin C, cDNA clone IMAGE:2821375)” in 2003. The replacement sequence has been deposited with GenBank and identified with accession number BC000449 or version BC000449.2 (as well as GI: 38197156). The sequence is 2201 nucleotides long and is as follows.

| | |
|---|---|
| 1 | cgggatttgg gtcgcggttc ttgtttgtgg atcgctgtga tcgtcacttg acaatgcaga |
| 61 | tcttcgtgaa gactctgact ggtaagacca tcaccctcga ggttgagccc agtgacacca |
| 121 | tcgagaatgt caaggcaaag atccaagata aggaaggcat ccctcctgac cagcagaggc |
| 181 | tgatctttgc tggaaaacag ctggaagatg ggcgcaccct gtctgactac aacatccaga |
| 241 | aagagtccac cctgcacctg gtgctccgtc tcagaggtgg gatgcaaatc ttcgtgaaga |
| 301 | cactcactgg caagaccatc acccttgagg tggagcccag tgacaccatc gagaacgtca |
| 361 | aagcaaagat ccaggacaag gaaggcattc ctcctgacca gcagaggttg atctttgccg |
| 421 | gaaagcagct ggaagatggg cgcaccctgt ctgactacaa catccagaaa gagtctaccc |
| 481 | tgcacctggt gctccgtctc agaggtggga tgcagatctt cgtgaagacc ctgactggta |
| 541 | agaccatcac cctcgaggtg gagcccagtg acaccatcga gaatgtcaag gcaaagatcc |
| 601 | aagataagga aggcattcct cctgatcagc agaggttgat ctttgccgga aaacagctgg |
| 661 | aagatggtcg taccctgtct gactacaaca tccagaaaga gtccaccttg cacctggtac |
| 721 | tccgtctcag aggtgggatg caaatcttcg tgaagacact cactggcaag accatcaccc |
| 781 | ttgaggtcga gcccagtgac actatcgaga acgtcaaagc aaagatccaa gacaaggaag |
| 841 | gcattcctcc tgaccagcag aggttgatct ttgccggaaa gcagctggaa gatgggcgca |
| 901 | ccctgtctga ctacaacatc cagaaagagt ctaccctgca cctggtgctc cgtctcagag |
| 961 | gtgggatgca gatcttcgtg aagaccctga ctggtaagac catcaccctc gaagtggagc |
| 1021 | cgagtgacac cattgagaat gtcaaggcaa agatccaaga caaggaaggc atccctcctg |
| 1081 | accagcagag gttgatcttt gccggaaaac agctggaaga tggtcgtacc ctgtctgact |
| 1141 | acaacatcca gaaagagtcc accttgcacc tggtgctccg tctcagaggt gggatgcaga |
| 1201 | tcttcgtgaa gaccctgact ggtaagacca tcactctcga ggtggagccg agtgacacca |
| 1261 | ttgagaatgt caaggcaaag atccaagaca aggaaggcat ccctcctgat cagcagaggt |
| 1321 | tgatctttgc tgggaaacag ctggaagatg gacgcaccct gtctgactac aacatccaga |
| 1381 | aagagtccac cctgcacctg gtgctccgtc ttagaggtgg gatgcagatc ttcgtgaaga |
| 1441 | ccctgactgg taagaccatc actctcgaag tggagccgag tgacaccatt gagaatgtca |
| 1501 | aggcaaagat ccaagacaag gaaggcatcc ctcctgacca gcagaggttg atctttgctg |
| 1561 | ggaaacagct ggaagatgga cgcaccctgt ctgactacaa catccagaaa gagtccaccc |
| 1621 | tgcacctggt gctccgtctt agaggtggga tgcagatctt cgtgaagacc ctgactggta |
| 1681 | agaccatcac tctcgaagtg gagccgagtg acaccattga gaatgtcaag gcaaagatcc |
| 1741 | aagacaagga aggcatccct cctgaccagc agaggttgat ctttgctggg aaacagctgg |
| 1801 | aagatggacg caccctgtct gactacaaca tccagaaaga gtccaccctg cacctggtgc |
| 1861 | tccgtctcag aggtgggatg cagatcttcg tgaagaccct gactggtaag accatcaccc |
| 1921 | tcgaggtgga gcccagtgac accatcgaga atgtcaaggc aaagatccaa gataaggaag |
| 1981 | gcatccctcc tgatcagcag aggttgatct ttgctgggaa acagctggaa gatggacgca |
| 2041 | ccctgtctga ctacaacatc cagaaagagt ccactctgca cttggtcctg cgcttgaggg |
| 2101 | ggggtgtcta agtttcccct tttaaggttt caacaaattt cattgcactt tcctttcaat |
| 2161 | aaagttgttg cattcccaaa aaaaaaaaaa aaaaaaaaaa a |

The underlined portion in each of the above is a 82 nucleotide long amplicon that is practiced in accordance with the instant invention. The amplicon is amplified by a forward Q-PCR primer having the sequence GGGTGTCTAAGTTTCCCCTTTTAAG and a reverse primer having the sequence TTTTTTGGGAATGCAACAACTTT.

This example is also exemplary of situations where the sequence to be detected is within a region less than about 100-150 nucleotides from the site of polyadenylation. The amplified sequence may be viewed as being about 76 nucleotides from the polyadenylation site.

### Example 3

The human sequence referred to as “succinate dehydrogenase complex, subunit A, flavoprotein (Fp), clone MGC: 1484 IMAGE:3051442” is expressed in many cell types. The sequence has been deposited with GenBank and identified with accession number BC001380 or version BC001380.1 (as well as GI: 12655060). This deposited sequence is 2310 nucleotides long and is as follows:

| | |
|---|---|
| 1 | ggcacgaggg gcgggactgc gcggcggcaa cagcagacat gtcgggggtc cggggcctgt |
| 61 | cgcggctgct gagcgctcgg cgcctggcgc tggccaaggc gtggccaaca gtgttgcaaa |
| 121 | caggaacccg aggttttcac ttcactgttg atgggaacaa gagggcatct gctaaagttt |
| 181 | cagattccat ttctgctcag tatccagtag tggatcatga atttgatgca gtggtggtag |
| 241 | gcgctggagg ggcaggcttg cgagctgcat ttggcctttc tgaggcaggg tttaatacag |
| 301 | catgtgttac caagctgttt cctaccaggt cacacactgt tgcagcacag ggaggaatca |
| 361 | atgctgctct ggggaacatg gaggaggaca actggaggtg gcatttctac gacaccgtga |
| 421 | agggctccga ctggctgggg gaccaggatg ccatccacta catgacggag caggcccccg |
| 481 | ccgccgtggt cgagctagaa aattatggca tgccgtttag cagaactgaa gatgggaaga |
| 541 | tttatcagcg tgcatttggt ggacagagcc tcaagtttgg aaagggcggg caggcccatc |
| 601 | ggtgctgctg tgtggctgat cggactggcc actcgctatt gcacacctta tatggaaggt |
| 661 | ctctgcgata tgataccagc tattttgtgg agtattttgc cttggatctc ctgatggaga |
| 721 | atggggagtg ccgtggtgtc atcgcactgt gcatagagga cgggtccatc catcgcataa |
| 781 | gagcaaagaa cactgttgtt gccacaggag gctacgggcg cacctacttc agctgcacgt |
| 841 | ctgcccacac cagcactggc gacggcacgg ccatgatcac cagggcaggc cttccttgcc |
| 901 | aggacctaga gtttgttcag ttccacccca caggcatata tggtgctggt tgtctcatta |
| 961 | cggaaggatg tcgtggagag ggaggcattc tcattaacag tcaaggcgaa aggtttatgg |
| 1021 | agcgatacgc ccctgtcgcg aaggacctgg cgtctagaga tgtggtgtct cggtccatga |
| 1081 | ctctggagat ccgagaagga agaggctgtg gccctgagaa agatcacgtc tacctgcagc |
| 1141 | tgcaccacct acctccagag cagctggcca cgcgcctgcc tggcatttca gagacagcca |
| 1201 | tgatcttcgc tggcgtggac gtcacgaagg agccgatccc tgtcctcccc accgtgcatt |
| 1261 | ataacatggg cggcattccc accaactaca aggggcaggt cctgaggcac gtgaatggcc |
| 1321 | aggatcagat tgtgcccggc ctgtacgcct gtggggaggc cgcctgtgcc tcggtacatg |
| 1381 | gtgccaaccg cctcggggca aactcgctct tggacctggt tgtctttggt cgggcatgtg |
| 1441 | ccctgagcat cgaagagtca tgcaggcctg gagataaagt ccctccaatt aaaccaaacg |
| 1501 | ctggggaaga atctgtcatg aatcttgaca aattgagatt tgctgatgga agcataagaa |
| 1561 | catcggaact gcgactcagc atgcagaagt caatgcaaaa tcatgctgcc gtgttccgtg |
| 1621 | tgggaagcgt gttgcaagaa ggttgtggga aaatcagcaa gctctatgga gacctaaagc |
| 1681 | acctgaagac gttcgaccgg ggaatggtct ggaacacgga cctggtggag accctggagc |
| 1741 | tgcagaacct gatgctgtgt gcgctgcaga ccatctacgg agcagaggca cggaaggagt |
| 1801 | cacggggcgc gcatgccagg gaagactaca aggtgcggat tgatgagtac gattactcca |
| 1861 | agcccatcca ggggcaacag aagaagccct ttgaggagca ctggaggaag cacaccctgt |
| 1921 | cctatgtgga cgttggcact gggaaggtca ctctggaata tagacccgtg atcgacaaaa |
| 1981 | ctttgaacga ggctgactgt gccaccgtcc cgccagccat tcgctcctac tgatgagaca |
| 2041 | agatgtggtg atgacagaat cagcttttgt aattatgtat aatagctcat gcatgtgtcc |
| 2101 | atgtcataac tgtcttcata cgcttctgca ctctggggaa gaaggagtac attgaaggga |
| 2161 | gattggcacc tagtggctgg gagcttgcca ggaacccagt ggccagggag cgtggcactt |
| 2221 | acctttgtcc cttgcttcat tcttgtgaga tgataaaact gggcacagct cttaaataaa |
| 2281 | atataaatga acaaaaaaaa aaaaaaaaaa |

This deposited sequence was replaced by a newer sequence referred to as “Homo sapiens succinate dehydrogenase complex, subunit A, flavoprotein (Fp), cDNA clone MGC:1484, IMAGE:3051442” in 2003. The replacement sequence has been deposited with GenBank and identified with accession number BC001380 or version BC001380.2 (as well as GI: 34783903). The sequence is 2301 nucleotides long and is as follows.

| | |
|---|---|
| 1 | ggcgggactg cgcggcggca acagcagaca tgtcgggggt ccggggcctg tcgcggctgc |
| 61 | tgagcgctcg gcgcctggcg ctggccaagg cgtggccaac agtgttgcaa acaggaaccc |
| 121 | gaggttttca cttcactgtt gatgggaaca agagggcatc tgctaaagtt tcagattcca |
| 181 | tttctgctca gtatccagta gtggatcatg aatttgatgc agtggtggta ggcgctggag |
| 241 | gggcaggctt gcgagctgca tttggccttt ctgaggcagg gtttaataca gcatgtgtta |
| 301 | ccaagctgtt tcctaccagg tcacacactg ttgcagcaca gggaggaatc aatgctgctc |
| 361 | tggggaacat ggaggaggac aactggaggt ggcatttcta cgacaccgtg aagggctccg |
| 421 | actggctggg ggaccaggat gccatccact acatgacgga gcaggccccc gccgccgtgg |
| 481 | tcgagctaga aaattatggc atgccgttta gcagaactga agatgggaag atttatcagc |
| 541 | gtgcatttgg tggacagagc ctcaagtttg gaaagggcgg gcaggcccat cggtgctgct |
| 601 | gtgtggctga tcggactggc cactcgctat tgcacacctt atatggaagg tctctgcgat |
| 661 | atgataccag ctattttgtg gagtattttg ccttggatct cctgatggag aatggggagt |
| 721 | gccgtggtgt catcgcactg tgcatagagg acgggtccat ccatcgcata agagcaaaga |
| 781 | acactgttgt tgcoacagga ggctacgggc gcacctactt cagctgcacg tctgcccaca |
| 841 | ccagcactgg cgacggcacg gccatgatca ccagggcagg ccttccttgc caggacctag |
| 901 | agtttgttca gttccacccc acaggcatat atggtgctgg ttgtctcatt acggaaggat |
| 961 | gtcgtggaga gggaggcatt ctcattaaca gtcaaggcga aaggtttatg gagcgatacg |
| 1021 | cccctgtcgc gaaggacctg gcgtctagag atgtggtgtc tcggtccatg actctggaga |
| 1081 | tccgagaagg aagaggctgt ggccctgaga aagatcacgt ctacctgcag ctgcaccacc |
| 1141 | tacctccaga gcagctggcc acgcgcctgc ctggcatttc agagacagcc atgatcttcg |
| 1201 | ctggcgtgga cgtcacgaag gagccgatcc ctgtcctccc caccgtgcat tataacatgg |
| 1261 | gcggcattcc caccaactac aaggggcagg tcctgaggca cgtgaatggc caggatcaga |
| 1321 | ttgtgcccgg cctgtacgcc tgtggggagg ccgcctgtgc ctcggtacat ggtgccaacc |
| 1381 | gcctcggggc aaactcgctc ttggacctgg ttgtctttgg tcgggcatgt gccctgagca |
| 1441 | tcgaagagtc atgcaggcct ggagataaag tccctccaat taaaccaaac gctggggaag |
| 1501 | aatctgtcat gaatcttgac aaattgagat ttgctgatgg aagcataaga acatcggaac |
| 1561 | tgcgactcag catgcagaag tcaatgcaaa atcatgctgc cgtgttccgt gtgggaagcg |
| 1621 | tgttgcaaga aggttgtggg aaaatcagca agctctatgg agacctaaag cacctgaaga |
| 1681 | cgttcgaccg gggaatggtc tggaacacgg acctggtgga gaccctggag ctgcagaacc |
| 1741 | tgatgctgtg tgcgctgcag accatctacg gagcagaggc acggaaggag tcacggggcg |
| 1801 | cgcatgccag ggaagactac aaggtgcgga ttgatgagta cgattactcc aagcccatcc |
| 1861 | aggggcaaca gaagaagccc tttgaggagc actggaggaa gcacaccctg tcctatgtgg |
| 1921 | acgttggcac tgggaaggtc actctggaat atagacccgt gatcgacaaa actttgaacg |
| 1981 | aggctgactg tgccaccgtc ccgccagcca ttcgctccta ctgatgagac aagatgtggt |
| 2041 | gatgacagaa tcagcttttg taattatgta taatagctca tgcatgtgtc catgtcataa |
| 2101 | ctgtcttcat acgcttctgc actctgggga agaaggagta cattgaaggg agattggcac |
| 2161 | ctagtggctg ggagcttgcc aggaacccag tggccaggga gcgtggcact tacctttgtc |
| 2221 | ccttgcttca ttcttgtgag atgataaaac tgggcacagc tcttaaataa aatataaatg |
| 2281 | aacaaaaaaa aaaaaaaaaa a |

The underlined portion in each of the above is a 60 nucleotide long amplicon that is practiced in accordance with the instant invention. The amplicon is amplified by a forward Q-PCR primer having the sequence GGGAGCGTGGCACTTACCT and a reverse primer having the sequence TGCCCAGTTTTATCATCTCACAA.

This example is also exemplary of situations where the sequence to be detected is within a region less than about 100-150 nucleotides from the site of polyadenylation. The amplified sequence may be viewed as being about 85 nucleotides from the polyadenylation site.

Indeed, this example has the detected sequence within about 20 or 30 nucleotides of the putative site of polyadenylation.

### Example 4

The Homo sapiens ribosomal protein L13a (RPL13A) sequence is expressed in many cell types. The sequence has been deposited with GenBank and identified with accession number NM_{—}012423 or version NM_{—}012423.2 (as well as GI:14591905). The deposited sequence is 1142 nucleotides long and is as follows:

| | |
|---|---|
| 1 | cttttccaag cggctgccga agatggcgga ggtgcaggtc ctggtgcttg atggtcgagg |
| 61 | ccatctcctg ggccgcctgg cggccatcgt ggctaaacag gtactgctgg gccggaaggt |
| 121 | ggtggtcgta cgctgtgaag gcatcaacat ttctggcaat ttctacagaa acaagttgaa |
| 181 | gtacctggct ttcctccgca agcggatgaa caccaaccct tcccgaggcc cctaccactt |
| 241 | ccgggccccc agccgcatct tctggcggac cgtgcgaggt atgctgcccc acaaaaccaa |
| 301 | gcgaggccag gccgctctgg accgtctcaa ggtgtttgac ggcatcccac cgccctacga |
| 361 | caagaaaaag cggatggtgg ttcctgctgc cctcaaggtc gtgcgtctga agcctacaag |
| 421 | aaagtttgcc tatctggggc gcctggctca cgaggttggc tggaagtacc aggcagtgac |
| 481 | agccaccctg gaggagaaga ggaaagagaa agccaagatc cactaccgga agaagaaaca |
| 541 | gctcatgagg ctacggaaac aggccgagaa gaacgtggag aagaaaattg acaaatacac |
| 601 | agaggtcctc aagacccacg gactcctggt ctgagcccaa taaagactgt taattcctca |
| 661 | tgcgttgcct gcccttcctc cattgttgcc ctggaatgta cgggacccag gggcagcagc |
| 721 | agtccaggtg ccacaggcag ccctgggaca taggaagctg ggagcaagga aagggtctta |
| 781 | gtcactgcct cccgaagttg cttgaaagca ctcggagaat tgtgcaggtg tcatttatct |
| 841 | atgaccaata ggaagagcaa ccagttacta tgagtgaaag ggagccagaa gactgattgg |
| 901 | agggccctat cttgtgagtg gggcatctgt tggactttcc acctggtcat atactctgca |
| 961 | gctgttagaa tgtgcaagca cttggggaca gcatgagctt gctgttgtac acagggtatt |
| 1021 | tctagaagca gaaatagact gggaagatgc acaaccaagg ggttacaggc atcgcccatg |
| 1081 | ctcctcacct gtattttgta atcagaaata aattgctttt aaagaaaaaa aaaaaaaaaa |
| 1141 | aa |

Position 1124 is identified as a putative polyadenylation site, and the underlined portion above is a 68 nucleotide long amplicon that is practiced in accordance with the instant invention. The amplicon is amplified by a forward Q-PCR primer having the sequence GGGAAGATGCACAACCAAGG and a reverse Q-PCR primer having the sequence TTTCTGATTACAAAATACAGGTGAGGA.

This example is exemplary of situations where the sequence to be detected is within a region less than about 100-150 nucleotides from the site of polyadenylation. Indeed, this example has the detected sequence within less than about 83 nucleotides from a putative site of polyadenylation.

All references cited herein are hereby incorporated by reference in their entireties, whether previously specifically incorporated or not. As used herein, the term “or” is intended to refer to alternatives and combinations.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

Citation of publications or documents herein is not intended as an admission that any is pertinent prior art. All statements as to the date or representation as to the contents of documents is based on the information available to the applicant and does not constitute any admission as to the correctness of the dates or contents of the documents.

1. A microarray comprising at least 5 oligonucleotide probes, each of 150 nucleotides or less in length, and complementary to at least 10 consecutive nucleotides of an mRNA molecule, wherein said at least 10 consecutive nucleotides is, in its entirety, less than 360 nucleotides from the site of poly(A) addition of said mRNA molecule.

2. The microarray of claim 1 comprising from at least 10 to 1000 probes wherein at least 10 probes are 150 nucleotides or less in length, and complementary to at least 10 consecutive nucleotides of an mRNA molecule, wherein said at least 10 consecutive nucleotides is, in its entirety, complementary to a sequence less than 360 nucleotides from the site of poly(A) addition of said mRNA molecule.

3. The microarray of claim 2 comprising at least 100 probes.

4. The microarray of claim 1 wherein said probes are complementary to at least 10 consecutive nucleotides is, in its entirety, less than 300 nucleotides from the site of poly(A) addition of said mRNA molecule.

5. The microarray of claim 1 wherein said probes are complementary to at least 20 consecutive nucleotides.

6. The microarray of claim 5 wherein said probes are complementary to at least 30 consecutive nucleotides.

7. The microarray of claim 4 wherein said probes are complementary to at least 20 consecutive nucleotides.

8. The microarray of claim 7 wherein said probes are complementary to at least 30 consecutive nucleotides.

9. A microarray comprising from 10 to 1000 oligonucleotide probes of 150 nucleotides or less, wherein at least 90% of the probes of said microarray are each complementary to at least 10 consecutive nucleotides of an mRNA molecule and wherein said at least 10 consecutive nucleotides is, in its entirety, less than 360 nucleotides from the site of poly(A) addition of said mRNA molecule.

10. The microarray of claim 9 comprising at least 100 probes.

11. The microarray of claim 9 wherein said probes are complementary to at least 10 consecutive nucleotides is, in its entirety, less than 300 nucleotides from the site of poly(A) addition of said mRNA molecule.

12. The microarray of claim 9 wherein said probes are complementary to at least 20 consecutive nucleotides.

13. The microarray of claim 12 wherein said probes are complementary to at least 30 consecutive nucleotides.

14. A microarray comprising less than 1000 oligonucleotide probes of 150 nucleotides or less, wherein at least 90% of said probes are each complementary to at least 10 consecutive nucleotides of an mRNA molecule and wherein said at least 10 consecutive nucleotides is, in its entirety, less than 360 nucleotides from the site of poly(A) addition of said mRNA molecule.

15. The microarray of claim 14 comprising at least 100 probes.

16. The microarray of claim 15 wherein said probes are complementary to at least 10 consecutive nucleotides is, in its entirety, less than 300 nucleotides from the site of poly(A) addition of said mRNA molecule.

17. The microarray of claim 14 wherein said probes are complementary to at least 20 consecutive nucleotides.

18. The microarray of claim 17 wherein said probes are complementary to at least 30 consecutive nucleotides.

19. The microarray of any one of claims 1-18 wherein said microarray is hybridized to RNA amplified from an FFPE sample.

20. A method of analyzing gene expression in a cell, comprising preparing a polynucleotide comprising gene sequences expressed in said cell and hybridizing said polynucleotide to a microarray according to any one of claims 1-18.

21. The method of claim 20 wherein said cell is from an FFPE sample.

22. The method of claim 20 or 21 wherein said cell is a breast cancer cell.

23. The method of any one of claims 20-22 wherein said cell has been isolated by microdissection of a cell containing sample.

24. A method of determining the presence of breast cancer in a subject, comprising preparing a polynucleotide comprising gene sequences expressed in a breast cancer cell of a cell containing sample from said subject, and hybridizing said amplified sequences to a microarray according to any one of claims 1-18.

25. The method of claim 24 wherein said sample is a biopsy.

26. The method of claim 24 wherein said sample is obtained by fine needle aspiration or ductal lavage.

27. The method of any one of claims 24-26 wherein said cell has been isolated by microdissection of said cell containing sample.

28. The microarray of claim 19 or the method of any one of claims 23-27 wherein said sample is a human sample.

29. A method of analyzing gene expression in a cell, comprising quantitative PCR amplification of a sequence containing at least 10 consecutive nucleotides of an mRNA molecule present in said cell,
wherein said at least 10 consecutive nucleotides is, in its entirety, less than 360 nucleotides from the site of poly(A) addition of said mRNA molecule and\
wherein said mRNA is optionally a reference sequence.

30. The method of claim 29 wherein said amplification is of an mRNA molecule obtained from said cell or of the corresponding cDNA.

31. The method of claim 29 wherein said cell is from an FFPE sample.

32. The method of claim 29 wherein said cell is a breast cancer cell.

33. The method of claim 29 wherein said cell has been isolated by microdissection of a cell containing sample.

34. A method of determining the presence of breast cancer in a subject, comprising quantitative PCR amplification of a sequence containing at least 10 consecutive nucleotides of an mRNA molecule present in a breast cancer cell of a cell containing sample from said subject, wherein said at least 10 consecutive nucleotides is, in its entirety, less than 360 nucleotides from the site of poly(A) addition of said mRNA molecule

35. The method of claim 34 wherein said sample is a biopsy.

36. The method of claim 34 wherein said sample is obtained by fine needle aspiration or ductal lavage.

37. The method of claim 34 wherein said cell has been isolated by microdissection of said cell containing sample.

38. The method of claim 29 wherein said cell is a human cell.

39. The method of claim 34 wherein said sample is a human sample.

40. The method of claim 34 further comprising the comparison of said quantitative PCR amplification of a sequence of said mRNA molecule with quantitative PCR amplification of a reference sequence from the same cell.
